(19) United States
(12) Patent Application Publication
(10) Pub. No.: US 2018/0134803 A1
(43) Pub. Date: May 17, 2018

(54) TREATMENT OF HER2-POSITIVE BREAST CANCER

(71) Applicants: Genentech, Inc., South San Francisco, CA (US); F. Hoffmann-La Roche AG, Basel (CH)

(72) Inventors: Hannah Douthwaite, Welwyn (GB); Maeve Waldron-Lynch, Welwyn (GB); Denise Bradley, Welwyn (GB); Jennifer Eng-Wong, South San Francisco, CA (US)

(21) Appl. No.: 15/801,937

(22) Filed: Nov. 2, 2017

Related U.S. Application Data
(60) Provisional application No. 62/417,966, filed on Nov. 4, 2016.

(51) Int. Cl. C07K 16/32; C07K 16/30; A61P 35/00; A61K 31/704; A61K 39/395; A61K 31/664; A61K 31/513; A61K 31/337

(52) U.S. Cl. CPC C07K 16/32; C07K 16/3015; A61P 35/00; A61K 31/704; A61K 2039/545; A61K 31/664; A61K 31/513; A61K 31/337; C07K 2317/24; A61K 39/3955

(57) ABSTRACT

Methods for the treatment of HER2-positive breast cancer are provided by neoadjuvant administration of pertuzumab and trastuzumab in combination with anthracycline-based chemotherapy. In particular, the methods concerns the treatment patients with HER2-positive, locally advanced, inflammatory, or early-stage breast cancer by neoadjuvant administration of pertuzumab and trastuzumab following anthracycline-based chemotherapy, wherein the combined administration of pertuzumab and trastuzumab increases pathological complete response (pCR) relative to administration of trastuzumab as a single agent, without significant increase in adverse events, such as cardiac toxicity, relative to neoadjuvant anthracycline-based chemotherapy.

### CROSS-REFERENCE TO RELATED APPLICATIONS

This application claims the benefit under 35 USC 119(e) of provisional U.S. Application No. 62/417,966, filed Nov. 4, 2016, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Sep. 26, 2017, is named GNE-0425-US_SL.txt and is 32,642 bytes in size.

### FIELD OF THE INVENTION

The present invention concerns the treatment of HER2-positive breast cancer by neoadjuvant administration of pertuzumab and trastuzumab in combination with anthracycline-based chemotherapy. In particular, the invention concerns the treatment patients with HER2-positive, locally advanced, inflammatory, or early-stage breast cancer by neoadjuvant administration of pertuzumab and trastuzumab following anthracycline-based chemotherapy, wherein the combined administration of pertuzumab and trastuzumab increases pathological complete response (pCR) relative to administration of trastuzumab as a single agent, without significant increase in adverse events, such as cardiac toxicity, relative to neoadjuvant anthracycline-based chemotherapy.

It also concerns an article of manufacture comprising a vial with pertuzumab therein and a package insert providing safety and/or efficacy data thereon; a method of making the article of manufacture; and a method of ensuring safe and effective use of pertuzumab in combination with trastuzumab related thereto.

### BACKGROUND OF THE INVENTION

Members of the HER family of receptor tyrosine kinases are important mediators of cell growth, differentiation and survival. The receptor family includes four distinct members including epidermal growth factor receptor (EGFR, ErbB1, or HER1), HER2 (ErbB2 or p185^{neu}), HER3 (ErbB3) and HER4 (ErbB4 or tyro2). Members of the receptor family have been implicated in various types of human malignancy.

A recombinant humanized version of the murine anti-HER2 antibody 4D5 (huMAb4D5-8, rhuMAb HER2, trastuzumab or HERCEPTN®; U.S. Pat. No. 5,821,337) is clinically active in patients with HER2-overexpressing metastatic breast cancers that have received extensive prior anti-cancer therapy (Baselga et al., J. Clin. Oncol. 14:737-744 (1996)).

Trastuzumab received marketing approval from the Food and Drug Administration Sep. 25, 1998 for the treatment of patients with metastatic breast cancer whose tumors overexpress the HER2 protein. At present, trastuzumab is approved for use as a single agent or in combination with chemotherapy or hormone therapy in the metastatic setting, and as single agent or in combination with chemotherapy as adjuvant treatment for patients with early-stage HER2-positive breast cancer. trastuzumab-based therapy is now the recommended treatment for patients with HER2-positive early-stage breast cancer who do not have contraindications for its use (Herceptin® prescribing information; NCCN Guidelines, version 2.2011). Trastuzumab plus docetaxel (or paclitaxel) is a registered standard of care in the first-line metastatic breast cancer (MBC) treatment setting (Slamon et al. N Engl J Med. 2001; 344(11):783-792; Marty et al. J Clin Oncol. 2005; 23(19):4265-4274).

Patients treated with the HER2 antibody trastuzumab are selected for therapy based on HER2 expression. See, for example, WO99/31140 (Paton et al.), US2003/0170234A1 (Hellmann, S.), and US2003/0147884 (Paton et al.); as well as WO01/89566, US2002/0064785, and US2003/0134344 (Mass et al.). See, also, U.S. Pat. No. 6,573,043, U.S. Pat. No. 6,905,830, and US2003/0152987, Cohen et al., concerning immunohistochemistry (IHC) and fluorescence in situ hybridization (FISH) for detecting HER2 overexpression and amplification. Thus, the optimal management of metastatic breast cancer now takes into account not only a patient's general condition, medical history, and receptor status, but also the HER2 status.

Pertuzumab (also known as recombinant humanized monoclonal antibody 2C4 (rhuMAb 2C4); Genentech, Inc, South San Francisco) represents the first in a new class of agents known as HER dimerization inhibitors (HDI) and functions to inhibit the ability of HER2 to form active heterodimers or homodimers with other HER receptors (such as EGFR/HER1, HER2, HER3 and HER4). See, for example, Harari and Yarden Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003).

Pertuzumab blockade of the formation of HER2-HER3 heterodimers in tumor cells has been demonstrated to inhibit critical cell signaling, which results in reduced tumor proliferation and survival (Agus et al. Cancer Cell 2:127-37 (2002)).

Pertuzumab has undergone testing as a single agent in the clinic with a phase Ia trial in patients with advanced cancers and phase II trials in patients with ovarian cancer and breast cancer as well as lung and prostate cancer. In a Phase I study, patients with incurable, locally advanced, recurrent or metastatic solid tumors that had progressed during or after standard therapy were treated with pertuzumab given intravenously every 3 weeks. Pertuzumab was generally well tolerated. Tumor regression was achieved in 3 of 20 patients evaluable for response. Two patients had confirmed partial responses. Stable disease lasting for more than 2.5 months was observed in 6 of 21 patients (Agus et al. Pro Am Soc Clin Oncol 22:192 (2003)). At doses of 2.0-15 mg/kg, the pharmacokinetics of pertuzumab was linear, and mean clearance ranged from 2.69 to 3.74 mL/day/kg and the mean terminal elimination half-life ranged from 15.3 to 27.6 days. Antibodies to pertuzumab were not detected (Allison et al. Pro Am Soc Clin Oncol 22:197 (2003)).

US 2006/0034842 describes methods for treating ErbB-expressing cancer with anti-ErbB2 antibody combinations. US 2008/0102069 describes the use of trastuzumab and pertuzumab in the treatment of HER2-positive metastatic cancer, such as breast cancer. Baselga et al., J Clin Oncol, 2007 ASCO Annual Meeting Proceedings Part I, Col. 25, No. 18S (June 20 Supplement), 2007:1004 report the treatment of patients with pre-treated HER2-positive breast cancer, which has progressed during treatment with trastuzumab, with a combination of trastuzumab and pertuzumab. Portera et al., J Clin Oncol, 2007 ASCO Annual Meeting Proceedings Part I. Vol. 25, No. 18S (June 20 Supplement), 2007:1028 evaluated the efficacy and safety of trastuzumab+pertuzumab combination therapy in HER2-positive breast cancer patients, who had progressive disease on trastuzumab-based therapy. The authors concluded that further evaluation of the efficacy of combination treatment was required to define the overall risk and benefit of this treatment regimen.

Pertuzumab has been evaluated in Phase II studies in combination with trastuzumab in patients with HER2-positive metastatic breast cancer who have previously received trastuzumab for metastatic disease. One study, conducted by the National cancer Institute (NCI), enrolled 11 patients with previously treated HER2-positive metastatic breast cancer. Two out of the 11 patients exhibited a partial response (PR) (Baselga et al., J Clin Oncol 2007 ASCO Annual Meeting Proceedings; 25:18S (June 20 Supplement): 1004).

The results of a Phase II neoadjuvant study evaluating the effect of a novel combination regimen of pertuzumab and trastuzumab plus chemotherapy (docetaxel) in women with early-stage HER2-positive breast cancer, presented at the CTRC-AACR San Antonio Breast Cancer Symposium (SABCS), Dec. 8-12, 2010, showed that the two HER2 antibodies plus docetaxel given in the neoadjuvant setting prior to surgery significantly improved the rate of complete tumor disappearance (pathological complete response rate, pCR, of 45.8 percent) in the breast by more than half compared to trastuzumab plus docetaxel (pCR of 29.0 percent), p=0.014.

The Clinical Evaluation of pertuzumab and trastuzumab (CLEOPATRA) Phase II clinical study assessed the efficacy and safety of pertuzumab plus trastuzumab plus docetaxel, as compared with placebo plus trastuzumab plus docetaxel, as first-line treatment for patients with locally recurrent, unresectable, or metastatic HER2-positive breast cancer. The combination of pertuzumab plus trastuzumab plus docetaxel, as compared with placebo plus trastuzumab plus docetaxel, when used as first-line treatment for HER2-positive metastatic breast cancer, significantly prolonged progression-free survival, with no increase in cardiac toxic effects. (Baselga et al., N Eng J Med 2012 366:2, 109-119).

The Phase II clinical study NeoSphere assessed the efficacy and safety of neoadjuvant administration of pertuzumab and trastuzumab in treatment-naïve women (patients who has not received any previous cancer therapy) with operable, locally advanced, and inflammatory breast cancer. Patients give pertuzumab and trastuzumab plus docetaxel showed a significantly improved pathological complete response rate compared with those given trastuzumab plus docetaxel, without substantial differences in tolerability (Gianni et al., Lancet Oncol 2012 13(1):25-32). Results of 5-year follow-up are reported by Gianni et al., Lancet Oncol 2016 17(6):791-800).

Patent Publications related to HER2 antibodies include: U.S. Pat. Nos. 5,677,171; 5,720,937; 5,720,954; 5,725,856; 5,770,195; 5,772,997; 6,165,464; 6,387,371; 6,399,063; 6,015,567; 6,333,169; 4,968,603; 5,821,337; 6,054,297; 6,407,213; 6,639,055; 6,719,971; 6,800,738; 5,648,237; 7,018,809; 6,267,958; 6,695,940; 6,821,515; 7,060,268; 7,682,609; 7,371,376; 6,127,526; 6,333,398; 6,797,814; 6,339,142; 6,417,335; 6,489,447; 7,074,404; 7,531,645; 7,846,441; 7,892,549; 6,573,043; 6,905,830; 7,129,840; 7,344,840; 7,468,252; 7,674,589; 6,949,245; 7,485,302; 7,498,030; 7,501,122; 7,537,931; 7,618,631; 7,862,817; 7,041,292; 6,627,196; 7,371,379; 6,632,979; 7,097,840; 7,575,748; 6,984,494; 7,279,287; 7,811,773; 7,993,834; 7,435,797; 7,850,966; 7,485,704; 7,807,799; 7,560,111; 7,879,325; 7,449,184; 7,700,299; and US 2010/0016556; US 2005/0244929; US 2001/0014326; US 2003/0202972; US 2006/0099201; US 2010/0158899; US 2011/0236383; US 2011/0033460; US 2005/0063972; US 2006/018739; US 2009/0220492; US 2003/0147884; US 2004/0037823; US 2005/0002928; US 2007/0292419; US 2008/0187533; US 2003/0152987; US 2005/0100944; US 2006/0183150; US2008/0050748; US 2010/0120053; US 2005/0244417; US 2007/0026001; US 2008/0160026; US 2008/0241146; US 2005/0208043; US 2005/0238640; US 2006/0034842; US 2006/0073143; US 2006/0193854; US 2006/0198843; US 2011/0129464; US 2007/0184055; US 2007/0269429; US 2008/0050373; US 2006/0083739; US 2009/0087432; US 2006/0210561; US 2002/0035736; US 2002/0001587; US 2008/0226659; US 2002/0090662; US 2006/0046270; US 2008/0108096; US 007/0166753; US 2008/0112958; US 2009/0239236; US 2004/008204; US 2009/0187007; US 2004/0106161; US 2011/0117096; US 2004/048525; US 2004/0258685; US 2009/0148401; US 2011/0117097; US 2006/0034840; US 2011/0064737; US 2005/0276812; US 2008/0171040; US 2009/0202536; US 2006/0013819; US 2006/0018899; US 2009/0285837; US 2011/0117097; US 2006/0088523; US 2010/0015157; US 2006/0121044; US 2008/0317753; US2006/0165702; US 2009/0081223; US 2006/0188509; US 2009/0155259; US 2011/0165157; US 2006/0204505; US 2006/0212956; US 2006/0275305; US 2007/0009976; US 2007/0020261; US 2007/0037228; US 2010/0112603; US 2006/0067930; US 2007/0224203; US 2008/0038271; US 2008/0050385; 2010/0285010; US 2008/0102069; US 2010/0008975; US 2011/0027190; US 2010/0298156; US 2009/0098135; US 2009/0148435; US 2009/0202546; US 2009/0226455; US 2009/0317387; and US 2011/0044977.

### SUMMARY OF THE INVENTION

New active treatments are required for patients with HER2-positive breast cancer, which is estimated to account for approximately 6000-8000 deaths per year in the United States, 12,000-15,000 deaths per year in Europe, and 60,000-90,000 deaths per year globally (based on mortality rates for breast cancer overall) (Levi et al., Eur J Cancer Prev 2005; 14:497-502; Estimates of worldwide burden of cancer in 2008: GLOBOCAN 2008. Int J Cancer 2010; 127:2893-917; SEER cancer statistics review, 1975-2008 [Internet]. Bethesda, Md. National Cancer Institute; November 2010 [updated, 2011]; Malvezzi et al., Ann Oncol 2013; 24:792-800). The median age of patients presenting with HER2-positive breast cancer is in the mid-50s, approximately 5 years younger than the general breast cancer population (Breast Cancer Res Treat 2008; 110:153-9; Breast Cancer Res 2009; 11:R31). At a time when the actuarial survival for women is >80 years of age, the median loss of life years per patient is approximately two decades. Improving the results of initial therapy when the disease is still localized to the breast and regional lymph nodes offers the chance of potentially curing the disease, as well as delaying disease recurrence and death in those who are not cured.

Promising results have been reported for combinations of PERJETA®, HERCEPTIN®, and chemotherapy in the neoadjuvant setting (Gianni et al., Lancet Oncol 2012; 13:25-32; Ann Oncol 2013; 24:2278-84), leading to the accelerated approval of PERJETA® for neoadjuvant use in the United States. Although a range of combination regimens have been evaluated, safety and efficacy data are needed on PERJETA®-containing neoadjuvant treatment regimens that include anthracycline-based chemotherapy and in particular treatment regimens including doxorubicin (rather than epirubicin) as the anthracycline, with dose-dense (dd) schedules, because such treatments and schedules are widely used in the adjuvant and neoadjuvant treatment of patients with breast cancer. In particular, doxorubicin plus cyclophosphamide (AC) followed by paclitaxel plus HERCEPTIN® (TH) is one of two preferred regimens for the neoadjuvant and adjuvant treatment of HER2-positive breast cancer according to NCCN guidelines.

More data are also required for PERJETA®-containing neoadjuvant regimens in which the chemotherapy component is given entirely prior to surgery, as opposed to splitting chemotherapy between neoadjuvant and adjuvant administration.

The present invention is based, at least in part, on the analysis of a non-randomized, open-label, multicenter, multinational Phase II clinical trial described in Example 1, which has been designed primarily to evaluate the cardiac safety of two neoadjuvant anthracycline/taxane-based regimens given in combination with PERJETA® and HERCEPTIN®.

In a first aspect, the invention concerns a method for the treatment of breast cancer comprising neoadjuvant administration to a patient with HER2-positive locally advanced, inflammatory, or early-stage breast cancer of an effective amount of a combination of pertuzumab and trastuzumab following anthracycline-based chemotherapy, wherein the combined administration of pertuzumab and trastuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.

In a particular aspect, the combined administration of pertuzumab and trastuzumab starts after at least 4 cycles of anthracycline-based chemotherapy.

In another aspect, the anthracycline-based chemotherapy comprises doxorubicin.

In a further aspect, the anthracycline-based chemotherapy comprises doxorubicin plus cyclophosphamide.

In a still further aspect, the anthracycline-based chemotherapy is doxorubicin plus cyclophosphamide (AC).

In a still further aspect, the anthracycline-based chemotherapy is dose-dense doxorubicin and cyclophosphamide (ddAC).

In an additional aspect, the doxorubicin plus cyclophosphamide are administered with G-CSF support.

In a particular aspect, the anthracycline-based chemotherapy is administered every two weeks.

In another aspect, at least four cycles of the anthracycline-based chemotherapy are administered prior to the combined administration of pertuzumab and trastuzumab.

In yet another aspect, the anthracycline-based chemotherapy comprises epirubicin.

In a further aspect, the anthracycline-based chemotherapy comprises epirubicin, 5-fluorouracil and cyclophosphamide.

In a still further aspect, the anthracycline-based chemotherapy is 5-fluorouracil, epirubicin plus cyclophosphamide (FEC).

In an additional aspect, the anthracycline-based chemotherapy is administered every three weeks.

In another aspect, at least four cycles of the anthracycline-based chemotherapy are administered prior to the combined administration of pertuzumab and trastuzumab.

In yet another aspect, pertuzumab and trastuzumab are administered in combination with neoadjuvant administration of a taxane, where the taxane may, for example, be docetaxel or paclitaxel.

In a further aspect, the combined administration of pertuzumab and trastuzumab starts at the start of taxane administration.

In a still further aspect, the pCR is breast pathological complete response (bpCR).

In an additional aspect, the pCR is total pathological complete response (tpCR).

In another aspect, the adverse events include cardiac side-effects.

In a particular aspect, the adverse event is a cardiac side-effect.

In one aspect, the cardiac side-effect comprises left ventricular ejection fraction (LVEF) drop.

In another aspect, the LVEF drop is asymptomatic.

In yet another aspect, the cardiac side-effect comprises left ventricular systolic dysfunction (LVSD).

In one aspect, the LVSD is symptomatic.

In another aspect, the HER2-positive breast cancer is characterized by immunohistochemistry (IHC) score 3+ or 2+ or by an amplification ratio of ≥2.0 determined by fluorescence in situ hybridization.

In a further aspect, the HER2-positive breast cancer is of Luminal A, Luminal B, HER2-Enriched (HER2-E) or Basal-like subtype as determined by PAM50 RT-qPCR assay.

In a still further aspect, the HER2-positive breast cancer is HER2-E subtype.

In an additional aspect, the HER2-positive breast cancer is characterized by aberrant PI3K pathway.

In a different aspect, the HER2-positive breast cancer is acetyltanshinone IIA (ATA) positive.

In a further aspect, the neoadjuvant administration is followed by definitive surgery.

In a still further aspect, definitive surgery is performed after at least eight cycles of neoadjuvant therapy.

In another aspect, definitive surgery is followed by adjuvant administration of pertuzumab plus trastuzumab.

In yet another aspect, pCR correlates with progression-free survival (PFS).

In a further aspect, the invention is directed to a method for extending the pathological complete response (pCR) in a patient with HER2-positive, locally advanced, inflammatory, or early-stage breast cancer by neoadjuvant administration of a combination of pertuzumab and trastuzumab following anthracycline-based chemotherapy, relative to administration of trastuzumab following anthracycline-containing chemotherapy, without significant increase in in adverse events relative to neoadjuvant anthracycline-containing chemotherapy.

In a different aspect, the invention concerns an article of manufacture comprising a vial with pertuzumab and a package insert, wherein the package insert provides at least part of the safety data shown in FIGS. 10-15.

In one aspect, the article of manufacture comprises a single-dose vial containing about 420 mg of pertuzumab.

In a different aspect, the invention concerns a method for making an article of manufacture comprising packaging together a vial with pertuzumab therein and a package insert, wherein the package insert provides at least part of the safety data shown in FIGS. 10-15.

The invention further concerns use of pertuzumab in the preparation of a medicament for treatment of breast cancer in a patient with HER2-positive locally advanced, inflammatory, or early-stage breast cancer comprising neoadjuvant administration of an effective amount of a combination of pertuzumab and trastuzumab following anthracycline-based chemotherapy, wherein the combined administration of pertuzumab and trastuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.

In another aspect, the invention concerns pertuzumab for use in the treatment of breast cancer in a patient with HER2-positive locally advanced, inflammatory, or early-stage breast cancer, wherein the treatment comprises neoadjuvant administration of an effective amount of a combination of pertuzumab and trastuzumab following anthracycline-based chemotherapy, wherein the combined administration of pertuzumab and trastuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.

In yet another aspect, the invention concerns use of trastuzumab in the preparation of a medicament for treatment of breast cancer in a patient with HER2-positive locally advanced, inflammatory, or early-stage breast cancer comprising neoadjuvant administration of an effective amount of a combination of trastuzumab and pertuzumab following anthracycline-based chemotherapy, wherein the combined administration of pertuzumab and trastuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.

In a further aspect, the invention concerns trastuzumab for use in the treatment of breast cancer in a patient with HER2-positive locally advanced, inflammatory, or early-stage breast cancer, wherein the treatment comprises neoadjuvant administration of an effective amount of a combination of trastuzumab and pertuzumab following anthracycline-based chemotherapy, wherein the combined administration of trastuzumab and pertuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.

### BRIEF DESCRIPTION OF THE DRAWINGS

FIG. 1 provides a schematic of the HER2 protein structure, and amino acid sequences for Domains I-IV (SEQ ID Nos.1-4, respectively) of the extracellular domain thereof.

FIGS. 2A and 2B depict alignments of the amino acid sequences of the variable light (V_{L}) (FIG. 2A) and variable heavy (V_{H}) (FIG. 2B) domains of murine monoclonal antibody 2C4 (SEQ ID Nos. 5 and 6, respectively); V_{L} and V_{H} domains of variant 574/pertuzumab (SEQ ID NOs. 7 and 8, respectively), and human V_{L} and V_{H} consensus frameworks (hum κ1, light kappa subgroup I; humIII, heavy subgroup III) (SEQ ID Nos. 9 and 10, respectively). Asterisks identify differences between variable domains of pertuzumab and murine monoclonal antibody 2C4 or between variable domains of pertuzumab and the human framework. Complementarity Determining Regions (CDRs) are in brackets.

FIGS. 3A and 3B show the amino acid sequences of pertuzumab light chain (FIG. 3A; SEQ ID NO. 11) and heavy chain (FIG. 3B; SEQ ID No. 12). CDRs are shown in bold. Calculated molecular mass of the light chain and heavy chain are 23,526.22 Da and 49,216.56 Da (cysteines in reduced form). The carbohydrate moiety is attached to Asn 299 of the heavy chain.

FIGS. 4A and 4B show the amino acid sequences of trastuzumab light chain (FIG. 4A; SEQ ID NO. 13) and heavy chain (FIG. 4B; SEQ ID NO. 14), respectively. Boundaries of the variable light and variable heavy domains are indicated by arrows.

FIGS. 5A and 5B depict a variant pertuzumab light chain sequence (FIG. 5A; SEQ ID NO. 15) and a variant pertuzumab heavy chain sequence (FIG. 5B; SEQ ID NO. 16), respectively.

FIG. 6 illustrates the study design of a Phase II clinical study to evaluate neoadjuvant administration of PERJETA® in combination with HERCEPTIN® and standard neoadjuvant anthracycline-based chemotherapy in patients with HER2-positive, locally advanced, inflammatory, or early-stage breast cancer described in Example 1. D=docetaxel; ddAC=dose-dense doxorubicin and cyclophosphamide; FEC=5-fluorouracil, epirubicin, cyclophosphamide; H=HERCEPTIN®; P=PERJETA®; T=paclitaxel.

FIG. 7 illustrates the screening procedure to identify eligible patients for the Phase II clinical study described in Example 1. CISH=chromogenic in situ hybridization; HER2=human epidermal growth factor receptor 2; IHC=immunohistochemistry; FISH=fluorescence in situ hybridization; SISH=silver in situ hybridization. HER2 positivity by central testing is defined as the following: IHC 3+ in >10% of immunoreactive cells or HER2 gene amplification by ISH (ratio of HER2 gene signals to centromere 17 signals ≥2.0).

FIG. 8 Algorithm for Continuation and Discontinuation of HER2-Targeted Study Medication.

FIG. 9 New York Heart Association (NYHA) Functional Classification System for Heart Failure.

FIG. 10 Summary of Adverse Events (AEs) During Neoadjuvant Treatment, Safety Population.

FIG. 11 Selected adverse events (AEs): Heart Failure (all classes), Neoadjuvant Period.

FIG. 12 Summary Table of Sustained Left Ventricular Ejection Fraction (LVEF) Declines.

FIG. 13 Most Common Serious Adverse Events (SAEs) during Neoadjuvant Treatment: Safety Population (All Grades). Incidence ≥2% in either Cohort.

FIG. 14 Most Common Adverse Events (AEs) during Neoadjuvant Treatment: Safety Population: Grade 3-5. Incidence ≥5% in either Cohort.

FIG. 15 Most Common AEs during Neoadjuvant Treatment: Safety Population (All Grades). Incidence ≥25% in either Cohort.

FIG. 16 Summary of total pathologic Complete Responses (tpCR) (local pathologist assessment).

FIG. 17 German Breast Study Group (GBG) pathological Complete Response (pCR) by tumor/nodal staging (TO NO): Intention-To-Treat (ITT) population.

FIG. 18 Total pathologic Complete Response (tpCR) by Cycles of Neoadjuvant Treatment (by tumor and nodal staging): Intention-To-Treat (ITT) population.

### DETAILED DESCRIPTION OF THE PREFERRED EMBODIMENTS

### I. Definitions

“Survival” refers to the patient remaining alive, and includes overall survival (OS) as well as progression free survival (PFS).

“Overall survival” or “OS” refers to the patient remaining alive for a defined period of time, such as 1 year, 2 years, 5 years, 10 years, etc. from the time of diagnosis or treatment. For the purposes of the clinical trial described in the example, overall survival (OS) is defined as the time from the date of randomization of patient population to the date of death from any cause.

“Progression free survival” or “PFS” refers to the patient remaining alive, without the cancer progressing or getting worse. For the purpose of the clinical trial described in the example, progression free survival (PFS) is defined as the time from randomization of study population to the first documented progressive disease, or unmanageable toxicity, or death from any cause, whichever occurs first. Disease progression can be documented by any clinically accepted methods, such as, for example, radiographical progressive disease, as determined by Response Evaluation Criteria in Solid Tumors (RECIST) (Therasse et al., J Natl Ca Inst 2000; 92(3):205-216), carcinomatous meningitis diagnosed by cytologic evaluation of cerebral spinal fluid, and/or medical photography to monitor chest wall recurrences of subcutaneous lesions.

By “extending survival” is meant increasing overall or progression free survival in a patient treated in accordance with the present invention relative to an untreated patient and/or relative to a patient treated with one or more approved anti-tumor agents, but not receiving treatment in accordance with the present invention. In a particular example, “extending survival” means extending progression-free survival (PFS) and/or overall survival (OS) of cancer patients receiving the combination therapy of the present invention (e.g. treatment with a combination of pertuzumab, trastuzumab and a chemotherapy) relative to patients treated with trastuzumab and the chemotherapy only. In another particular example, “extending survival” means extending progression-free survival (PFS) and/or overall survival (OS) of cancer patients receiving the combination therapy of the present invention (e.g. treatment with a combination of pertuzumab, trastuzumab and a chemotherapy) relative to patients treated with pertuzumab and the chemotherapy only.

An “objective response” (OR) refers to a measurable response, including complete response (CR) or partial response (PR).

By “complete response” or “CR” is intended the disappearance of all signs of cancer in response to treatment. This does not always mean the cancer has been cured.

“Partial response” or “PR” refers to a decrease in the size of one or more tumors or lesions, or in the extent of cancer in the body, in response to treatment.

A “HER receptor” is a receptor protein tyrosine kinase which belongs to the HER receptor family and includes EGFR, HER2, HER3 and HER4 receptors. The HER receptor will generally comprise an extracellular domain, which may bind an HER ligand and/or dimerize with another HER receptor molecule; a lipophilic transmembrane domain; a conserved intracellular tyrosine kinase domain; and a carboxyl-terminal signaling domain harboring several tyrosine residues which can be phosphorylated. The HER receptor may be a “native sequence” HER receptor or an “amino acid sequence variant” thereof. Preferably the HER receptor is native sequence human HER receptor.

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363). The term “erbB2” refers to the gene encoding human ErbB2 and “neu” refers to the gene encoding rat p185^{neu}. Preferred HER2 is native sequence human HER2.

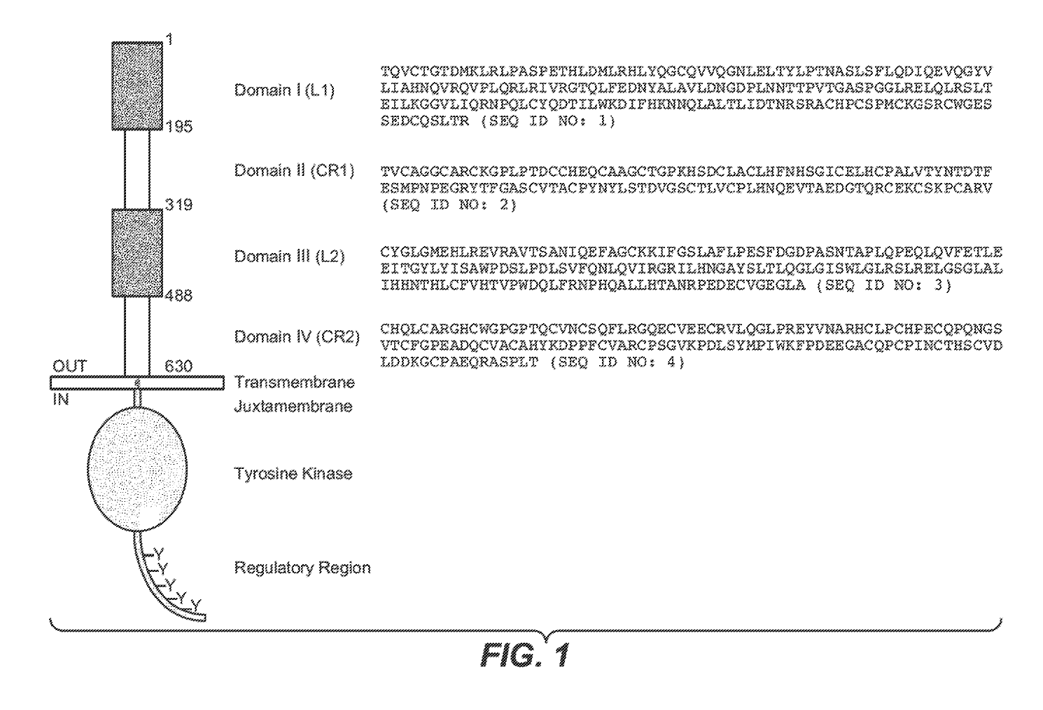

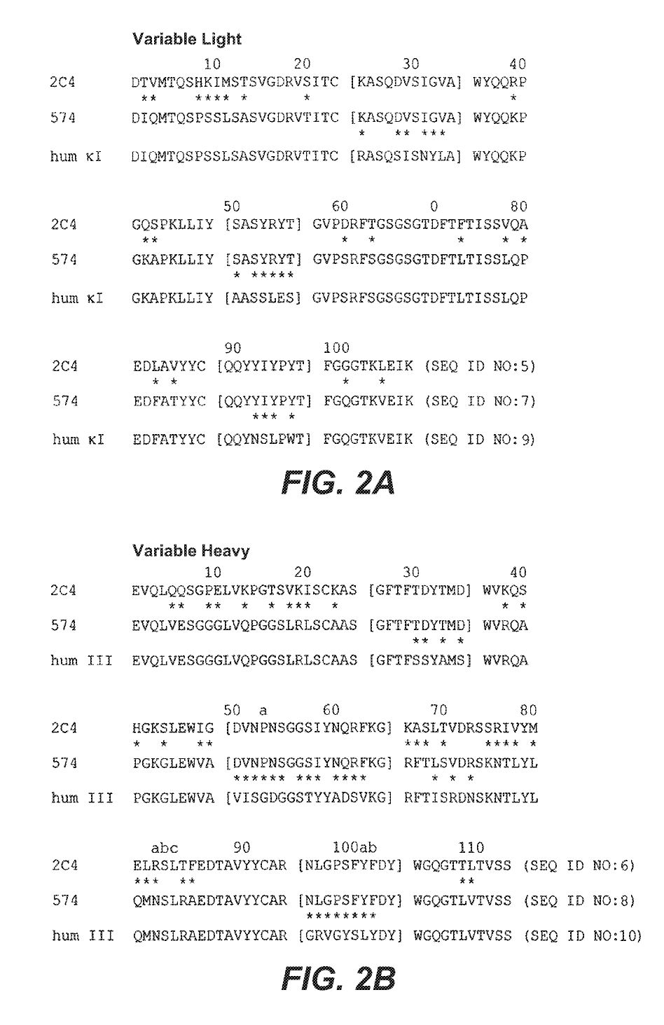

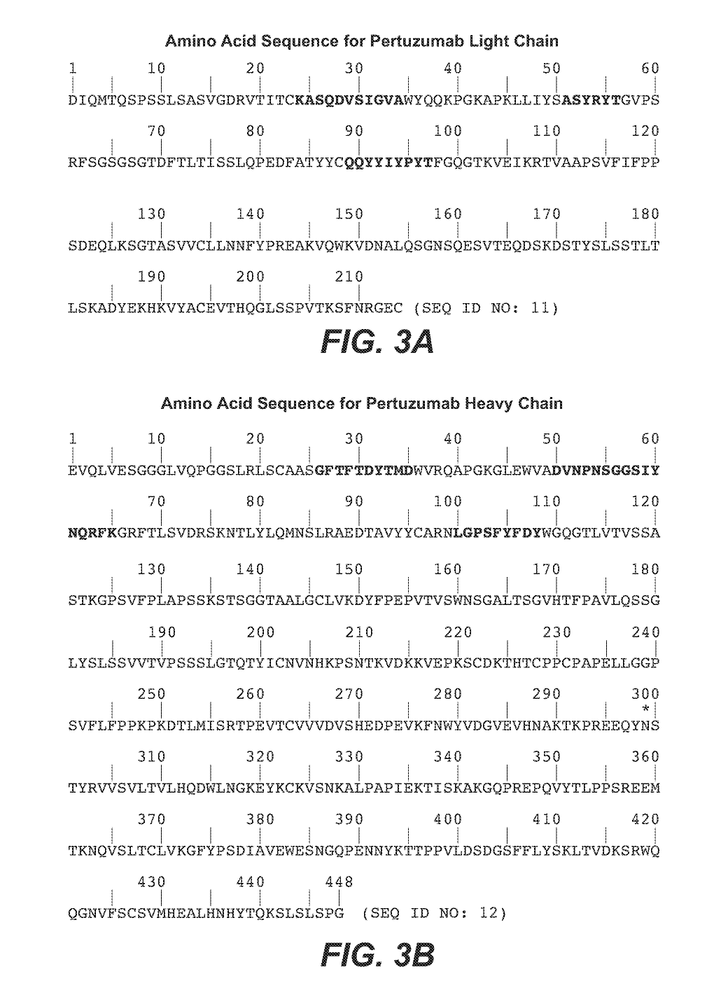

Herein, “HER2 extracellular domain” or “HER2 ECD” refers to a domain of HER2 that is outside of a cell, either anchored to a cell membrane, or in circulation, including fragments thereof. The amino acid sequence of HER2 is shown in FIG. 1. In one embodiment, the extracellular domain of HER2 may comprise four domains: “Domain I” (amino acid residues from about 1-195; SEQ ID NO:1), “Domain II” (amino acid residues from about 196-319; SEQ ID NO:2), “Domain III” (amino acid residues from about 320-488: SEQ ID NO:3), and “Domain IV” (amino acid residues from about 489-630; SEQ ID NO:4) (residue numbering without signal peptide). See Garrett et al. Mol. Cell. 11: 495-505 (2003), Cho et al. Nature 421: 756-760 (2003), Franklin et al. Cancer Cell 5:317-328 (2004), and Plowman et al. Proc. Natl. Acad. Sci. 90:1746-1750 (1993), as well as FIG. 1 herein.

“HER3” or “ErbB3” herein refer to the receptor as disclosed, for example, in U.S. Pat. Nos. 5,183,884 and 5,480,968 as well as Kraus et al. PNAS (USA) 86:9193-9197 (1989).

A “low HER3” cancer is one which expresses HER3 at a level less than the median level for HER3 expression in the cancer type. In one embodiment, the low HER3 cancer is epithelial ovarian, peritoneal, or fallopian tube cancer. HER3 DNA, protein, and/or mRNA level in the cancer can be evaluated to determine whether the cancer is a low HER3 cancer. See, for example, U.S. Pat. No. 7,981,418 for additional information about low HER3 cancer. Optionally, a HER3 mRNA expression assay is performed in order to determine that the cancer is a low HER3 cancer. In one embodiment, HER3 mRNA level in the cancer is evaluated, e.g. using polymerase chain reaction (PCR), such as quantitative reverse transcription PCR (qRT-PCR). Optionally, the cancer expresses HER3 at a concentration ratio equal or lower than about 2.81 as assessed qRT-PCR, e.g. using a COBAS z480® instrument.

A “HER dimer” herein is a noncovalently associated dimer comprising at least two HER receptors. Such complexes may form when a cell expressing two or more HER receptors is exposed to an HER ligand and can be isolated by immunoprecipitation and analyzed by SDS-PAGE as described in Sliwkowski et al., J. Biol. Chem., 269(20):14661-14665 (1994), for example. Other proteins, such as a cytokine receptor subunit (e.g. gp130) may be associated with the dimer. Preferably, the HER dimer comprises HER2.

A “HER heterodimer” herein is a noncovalently associated heterodimer comprising at least two different HER receptors, such as EGFR-HER2, HER2-HER3 or HER2-HER4 heterodimers.

A “HER antibody” is an antibody that binds to a HER receptor. Optionally, the HER antibody further interferes with HER activation or function. Preferably, the HER antibody binds to the HER2 receptor. HER2 antibodies of interest herein are pertuzumab and trastuzumab.

“HER activation” refers to activation, or phosphorylation, of any one or more HER receptors. Generally, HER activation results in signal transduction (e.g. that caused by an intracellular kinase domain of a HER receptor phosphorylating tyrosine residues in the HER receptor or a substrate polypeptide). HER activation may be mediated by HER ligand binding to a HER dimer comprising the HER receptor of interest. HER ligand binding to a HER dimer may activate a kinase domain of one or more of the HER receptors in the dimer and thereby results in phosphorylation of tyrosine residues in one or more of the HER receptors and/or phosphorylation of tyrosine residues in additional substrate polypeptides(s), such as Akt or MAPK intracellular kinases.

“Phosphorylation” refers to the addition of one or more phosphate group(s) to a protein, such as a HER receptor, or substrate thereof.

An antibody which “inhibits HER dimerization” is an antibody which inhibits, or interferes with, formation of a HER dimer. Preferably, such an antibody binds to HER2 at the heterodimeric binding site thereof. The most preferred dimerization inhibiting antibody herein is pertuzumab or MAb 2C4. Other examples of antibodies which inhibit HER dimerization include antibodies which bind to EGFR and inhibit dimerization thereof with one or more other HER receptors (for example EGFR monoclonal antibody 806, MAb 806, which binds to activated or “untethered” EGFR; see Johns et al., J. Biol. Chem. 279(29):30375-30384 (2004)); antibodies which bind to HER3 and inhibit dimerization thereof with one or more other HER receptors; and antibodies which bind to HER4 and inhibit dimerization thereof with one or more other HER receptors.

A “HER2 dimerization inhibitor” is an agent that inhibits formation of a dimer or heterodimer comprising HER2.

A “heterodimeric binding site” on HER2, refers to a region in the extracellular domain of HER2 that contacts, or interfaces with, a region in the extracellular domain of EGFR, HER3 or HER4 upon formation of a dimer therewith. The region is found in Domain II of HER2 (SEQ ID NO: 15). Franklin et al. Cancer Cell 5:317-328 (2004).

A HER2 antibody that “binds to a heterodimeric binding site” of HER2, binds to residues in Domain II (SEQ ID NO: 2) and optionally also binds to residues in other of the domains of the HER2 extracellular domain, such as domains I and III, SEQ ID NOs: 1 and 3), and can sterically hinder, at least to some extent, formation of a HER2-EGFR, HER2-HER3, or HER2-HER4 heterodimer. Franklin et al. Cancer Cell 5:317-328 (2004) characterize the HER2-pertuzumab crystal structure, deposited with the RCSB Protein Data Bank (ID Code IS78), illustrating an exemplary antibody that binds to the heterodimeric binding site of HER2.

An antibody that “binds to domain II” of HER2 binds to residues in domain II (SEQ ID NO: 2) and optionally residues in other domain(s) of HER2, such as domains I and III (SEQ ID NOs: 1 and 3, respectively). Preferably the antibody that binds to domain II binds to the junction between domains I, II and III of HER2.

For the purposes herein, “pertuzumab” and “rhuMAb 2C4”, which are used interchangeably, refer to an antibody comprising the variable light and variable heavy amino acid sequences in SEQ ID NOs: 7 and 8, respectively. Where pertuzumab is an intact antibody, it preferably comprises an IgG1 antibody; in one embodiment comprising the light chain amino acid sequence in SEQ ID NO: 11 or 15, and heavy chain amino acid sequence in SEQ ID NO: 12 or 16. The antibody is optionally produced by recombinant Chinese Hamster Ovary (CHO) cells. The terms “pertuzumab” and “rhuMAb 2C4” herein cover biosimilar versions of the drug with the United States Adopted Name (USAN) or International Nonproprietary Name (INN): pertuzumab.

For the purposes herein, “trastuzumab” and “rhuMAb4D5”, which are used interchangeably, refer to an antibody comprising the variable light and variable heavy amino acid sequences from within SEQ ID Nos: 13 and 14, respectively. Where trastuzumab is an intact antibody, it preferably comprises an IgG1 antibody; in one embodiment comprising the light chain amino acid sequence of SEQ ID NO: 13 and the heavy chain amino acid sequence of SEQ ID NO: 14. The antibody is optionally produced by Chinese Hamster Ovary (CHO) cells. The terms “trastuzumab” and “rhuMAb4D5” herein cover biosimilar versions of the drug with the United States Adopted Name (USAN) or International Nonproprietary Name (INN): trastuzumab.

The term “antibody” herein is used in the broadest sense and specifically covers monoclonal antibodies, polyclonal antibodies, multispecific antibodies (e.g. bispecific antibodies), and antibody fragments, so long as they exhibit the desired biological activity.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). Humanized HER2 antibodies specifically include trastuzumab (HERCEPTIN®) as described in Table 3 of U.S. Pat. No. 5,821,337 expressly incorporated herein by reference and as defined herein; and humanized 2C4 antibodies such as pertuzumab as described and defined herein.

An “intact antibody” herein is one which comprises two antigen binding regions, and an Fc region. Preferably, the intact antibody has a functional Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays as herein disclosed, for example.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into Asubclasses@ (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

A “naked antibody” is an antibody that is not conjugated to a heterologous molecule, such as a cytotoxic moiety or radiolabel.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “deamidated” antibody is one in which one or more asparagine residues thereof has been derivitized, e.g. to an aspartic acid, a succinimide, or an iso-aspartic acid.

The terms “cancer” and “cancerous” refer to or describe the physiological condition in mammals that is typically characterized by unregulated cell growth.

“Gastric cancer” specifically includes metastatic or locally advanced non-resectable gastric cancer, including, without limitation, histologically confirmed adenocarcinoma of the stomach or gastroesophageal junction with inoperable (non-resectable) locally advanced or metastatic disease, not amenable to curative therapy, and post-operatively recurrent advanced gastric cancer, such as adenocarcinoma of the stomach or gastroesophageal junction, when the intent of the surgery was to cure the disease.

An “advanced” cancer is one which has spread outside the site or organ of origin, either by local invasion or metastasis. Accordingly, the term “advanced” cancer includes both locally advanced and metastatic disease.

A “refractory” cancer is one which progresses even though an anti-tumor agent, such as a chemotherapy, is being administered to the cancer patient. An example of a refractory cancer is one which is platinum refractory.

A “recurrent” cancer is one which has regrown, either at the initial site or at a distant site, after a response to initial therapy, such as surgery.

A “locally recurrent” cancer is cancer that returns after treatment in the same place as a previously treated cancer.

A “non-resectable” or “unresectable” cancer is not able to be removed (resected) by surgery.

“Early-stage breast cancer” herein refers to breast cancer that has not spread beyond the breast or the axillary lymph nodes. Such cancer is generally treated with neoadjuvant or adjuvant therapy.

“Neoadjuvant therapy” or “neoadjuvant treatment” or “neoadjuvant administration” refers to systemic therapy given prior to surgery.

“Adjuvant therapy” or “adjuvant treatment” or “adjuvant administration” refers to systemic therapy given after surgery.

“Metastatic” cancer refers to cancer which has spread from one part of the body (e.g. the breast) to another part of the body.

Herein, a “patient” or “subject” is a human patient. The patient may be a “cancer patient,” i.e. one who is suffering or at risk for suffering from one or more symptoms of cancer, in particular breast cancer.

A “patient population” refers to a group of cancer patients. Such populations can be used to demonstrate statistically significant efficacy and/or safety of a drug, such as pertuzumab and/or trastuzumab.

A “relapsed” patient is one who has signs or symptoms of cancer after remission. Optionally, the patient has relapsed after adjuvant or neoadjuvant therapy.

A cancer or biological sample which “displays HER expression, amplification, or activation” is one which, in a diagnostic test, expresses (including overexpresses) a HER receptor, has amplified HER gene, and/or otherwise demonstrates activation or phosphorylation of a HER receptor.

A cancer or biological sample which “displays HER activation” is one which, in a diagnostic test, demonstrates activation or phosphorylation of a HER receptor. Such activation can be determined directly (e.g. by measuring HER phosphorylation by ELISA) or indirectly (e.g. by gene expression profiling or by detecting HER heterodimers, as described herein).

A cancer cell with “HER receptor overexpression or amplification” is one which has significantly higher levels of a HER receptor protein or gene compared to a noncancerous cell of the same tissue type. Such overexpression may be caused by gene amplification or by increased transcription or translation. HER receptor overexpression or amplification may be determined in a diagnostic or prognostic assay by evaluating increased levels of the HER protein present on the surface of a cell (e.g. via an immunohistochemistry assay; IHC). Alternatively, or additionally, one may measure levels of HER-encoding nucleic acid in the cell, e.g. via in situ hybridization (ISH), including fluorescent in situ hybridization (FISH; see WO98/45479 published October, 1998) and chromogenic in situ hybridization (CISH; see, e.g. Tanner et al., Am. J. Pathol. 157(5): 1467-1472 (2000); Bella et al., J. Clin. Oncol. 26: (May 20 suppl; abstr 22147) (2008)), southern blotting, or polymerase chain reaction (PCR) techniques, such as quantitative real time PCR (qRT-PCR). One may also study HER receptor overexpression or amplification by measuring shed antigen (e.g., HER extracellular domain) in a biological fluid such as serum (see, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

A “HER2-positive” cancer comprises cancer cells which have higher than normal levels of HER2. Examples of HER2-positive cancer include HER2-positive breast cancer and HER2-positive gastric cancer. Optionally, HER2-positive cancer has an immunohistochemistry (IHC) score of 2+ or 3+ and/or an in situ hybridization (ISH) amplification ratio ≥2.0.

Herein, an “anti-tumor agent” refers to a drug used to treat cancer. Non-limiting examples of anti-tumor agents herein include chemotherapy agents, HER dimerization inhibitors, HER antibodies, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxane, dual tyrosine kinase inhibitors, TLK286, EMD-7200, pertuzumab, trastuzumab, erlotinib, and bevacizumab.

The “epitope 2C4” is the region in the extracellular domain of HER2 to which the antibody 2C4 binds. In order to screen for antibodies which bind essentially to the 2C4 epitope, a routine cross-blocking assay such as that described in Antibodies, A Laboratory Manual, Cold Spring Harbor Laboratory, Ed Harlow and David Lane (1988), can be performed. Preferably the antibody blocks 2C4's binding to HER2 by about 50% or more. Alternatively, epitope mapping can be performed to assess whether the antibody binds essentially to the 2C4 epitope of HER2. Epitope 2C4 comprises residues from Domain II (SEQ ID NO: 2) in the extracellular domain of HER2. 2C4 and pertuzumab binds to the extracellular domain of HER2 at the junction of domains I, II and III (SEQ ID NOs: 1, 2, and 3, respectively). Franklin et al. Cancer Cell 5:317-328 (2004).

The “epitope 4D5” is the region in the extracellular domain of HER2 to which the antibody 4D5 (ATCC CRL 10463) and trastuzumab bind. This epitope is close to the transmembrane domain of HER2, and within Domain IV of HER2 (SEQ ID NO: 4). To screen for antibodies which bind essentially to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds essentially to the 4D5 epitope of HER2 (e.g. any one or more residues in the region from about residue 529 to about residue 625, inclusive of the HER2 ECD, residue numbering including signal peptide).

“Treatment” refers to both therapeutic treatment and prophylactic or preventative measures. Those in need of treatment include those already with cancer as well as those in which cancer is to be prevented. Hence, the patient to be treated herein may have been diagnosed as having cancer or may be predisposed or susceptible to cancer.

The term “effective amount” refers to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI).

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapy” is use of a chemical compound useful in the treatment of cancer. Examples of chemotherapeutic agents, used in chemotherapy, include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)) and anthracyclines such as annamycin, AD 32, alcarubicin, daunorubicin, doxorubicin, dexrazoxane, DX-52-1, epirubicin, GPX-100, idarubicin, valrubicin, KRN5500, menogaril, dynemicin, including dynemicin A, an esperamicin, neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX^{RM}, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK7 polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxanes; chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “taxane” is a chemotherapy which inhibits mitosis and interferes with microtubules. Examples of taxanes include Paclitaxel (TAXOL®; Bristol-Myers Squibb Oncology, Princeton, N.J.); cremophor-free, albumin-engineered nanoparticle formulation of paclitaxel or nab-paclitaxel (ABRAXANE™; American Pharmaceutical Partners, Schaumberg, Ill.); and Docetaxel (TAXOTERE®; Rhone-Poulenc Rorer, Antony, France).

An “anthacycline” is a type of antibiotic that comes from the fungus Streptococcus peucetius, examples include: Daunorubicin, Doxorubicin, Epirubicin, and any other anthracycline chemotherapeutic agents, including those listed before.

“Anthracycline-based chemotherapy” refers to a chemotherapy regimen that consists of or includes one or more anthracycline. Examples include, without limitation, 5-FU, epirubicin, and cyclophosphamide (FEC); 5-FU, doxorubicin, and cyclophosphamide (FAC); doxorubicin and cyclophosphamide (AC); epirubicin and cyclophosphamide (EC); dose-dense doxorubicin and cyclophosphamide (ddAC), and the like.

For the purposes herein, “carboplatin-based chemotherapy” refers to a chemotherapy regimen that consists of or includes one or more carboplatins. An example is TCH (Docetaxel/TAXOL®, Carboplatin, and trastuzumab/HERCEPTIN®).

An “aromatase inhibitor” inhibits the enzyme aromatase, which regulates estrogen production in the adrenal glands. Examples of aromatase inhibitors include: 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In one embodiment, the aromatase inhibitor herein is letrozole or anastrozole.

An “antimetabolite chemotherapy” is use of an agent which is structurally similar to a metabolite, but cannot be used by the body in a productive manner. Many antimetabolite chemotherapy interferes with the production of the nucleic acids, RNA and DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOME®), azocytosine, deoxycytosine, pyridmidene, fludarabine (FLUDARA®), cladrabine, 2-deoxy-D-glucose etc.

By “chemotherapy-resistant” cancer is meant that the cancer patient has progressed while receiving a chemotherapy regimen (i.e. the patient is “chemotherapy refractory”), or the patient has progressed within 12 months (for instance, within 6 months) after completing a chemotherapy regimen.

The term “platin” is used herein to refer to platinum based chemotherapy, including, without limitation, cisplatin, carboplatin, and oxaliplatin.

The term “fluoropyrimidine” is used herein to refer to an antimetabolite chemotherapy, including, without limitation, capecitabine, floxuridine, and fluorouracil (5-FU).

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks, preferably every 3 weeks.

“Infusion” or “infusing” refers to the introduction of a drug-containing solution into the body through a vein for therapeutic purposes. Generally, this is achieved via an intravenous (IV) bag.

An “intravenous bag” or “IV bag” is a bag that can hold a solution which can be administered via the vein of a patient. In one embodiment, the solution is a saline solution (e.g. about 0.9% or about 0.45% NaCl). Optionally, the IV bag is formed from polyolefin or polyvinyl chloride.

By “co-administering” is meant intravenously administering two (or more) drugs during the same administration, rather than sequential infusions of the two or more drugs. Generally, this will involve combining the two (or more) drugs into the same IV bag prior to co-administration thereof.

“Cardiac toxicity” refers to any toxic side effect resulting from administration of a drug or drug combination. Cardiac toxicity can be evaluated based on any one or more of: incidence of symptomatic left ventricular systolic dysfunction (LVSD) or congestive heart failure (CHF), or decrease in left ventricular ejection fraction (LVEF).

The phrase “without increasing cardiac toxicity” for a drug combination including pertuzumab refers to an incidence of cardiac toxicity that is equal or less than that observed in patients treated with drugs other than pertuzumab in the drug combination (e.g. equal or less than that resulting from administration of trastuzumab and the chemotherapy, e.g. docetaxel).

A “vial” is a container suitable for holding a liquid or lyophilized preparation. In one embodiment, the vial is a single-use vial, e.g. a 20-cc single-use vial with a stopper.

A “package insert” is a leaflet that, by order of the Food and Drug Administration (FDA) or other Regulatory Authority, must be placed inside the package of every prescription drug. The leaflet generally includes the trademark for the drug, its generic name, and its mechanism of action; states its indications, contraindications, warnings, precautions, adverse effects, and dosage forms; and includes instructions for the recommended dose, time, and route of administration.

The expression “safety data” concerns the data obtained in a controlled clinical trial showing the prevalence and severity of adverse events to guide the user regarding the safety of the drug, including guidance on how to monitor and prevent adverse reactions to the drug. Table 3 and Table 4 herein provide safety data for pertuzumab. The safety data comprises any one or more (e.g. two, three, four or more) of the most common adverse events (AEs) or adverse reactions (ADRs) in Tables 3 and 4. For example, the safety data comprises information about neutropenia, febrile neutropenia, diarrhea and/or cardiac toxicity as disclosed herein.

“Efficacy data” refers to the data obtained in controlled clinical trial showing that a drug effectively treats a disease, such as cancer.

By “stable mixture” when referring to a mixture of two or more drugs, such as pertuzumab and trastuzumab” means that each of the drugs in the mixture essentially retains its physical and chemical stability in the mixture as evaluated by one or more analytical assays. Exemplary analytical assays for this purpose include: color, appearance and clarity (CAC), concentration and turbidity analysis, particulate analysis, size exclusion chromatography (SEC), ion-exchange chromatography (IEC), capillary zone electrophoresis (CZE), image capillary isoelectric focusing (iCIEF), and potency assay. In one embodiment, mixture has been shown to be stable for up to 24 hours at 5° C. or 30° C.

A drug that is administered “concurrently” with one or more other drugs is administered during the same treatment cycle, on the same day of treatment as the one or more other drugs, and, optionally, at the same time as the one or more other drugs. For instance, for cancer therapies given every 3-weeks, the concurrently administered drugs are each administered on day-1 of a 3-week cycle.

### II. Antibody and Chemotherapy Compositions

The HER2 antigen to be used for production of antibodies may be, e.g., a soluble form of the extracellular domain of a HER2 receptor or a portion thereof, containing the desired epitope. Alternatively, cells expressing HER2 at their cell surface (e.g. NIH-3T3 cells transformed to overexpress HER2; or a carcinoma cell line such as SK-BR-3 cells, see Stancovski et al. PNAS (USA) 88:8691-8695 (1991)) can be used to generate antibodies. Other forms of HER2 receptor useful for generating antibodies will be apparent to those skilled in the art.

Various methods for making monoclonal antibodies herein are available in the art. For example, the monoclonal antibodies may be made using the hybridoma method first described by Kohler et al., Nature, 256:495 (1975), by recombinant DNA methods (U.S. Pat. No. 4,816,567).

The anti-HER2 antibodies used in accordance with the present invention, trastuzumab and pertuzumab, are commercially available.

(i) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

U.S. Pat. No. 6,949,245 describes production of exemplary humanized HER2 antibodies which bind HER2 and block ligand activation of a HER receptor.

Humanized HER2 antibodies specifically include trastuzumab as described in Table 3 of U.S. Pat. No. 5,821,337 expressly incorporated herein by reference and as defined herein; and humanized 2C4 antibodies such as pertuzumab as described and defined herein.

The humanized antibodies herein may, for example, comprise nonhuman hypervariable region residues incorporated into a human variable heavy domain and may further comprise a framework region (FR) substitution at a position selected from the group consisting of 69H, 71H and 73H utilizing the variable domain numbering system set forth in Kabat et al., Sequences of Proteins of Immunological Interest, 5th Ed. Public Health Service, National Institutes of Health, Bethesda, Md. (1991). In one embodiment, the humanized antibody comprises FR substitutions at two or all of positions 69H, 71H and 73H.

An exemplary humanized antibody of interest herein comprises variable heavy domain complementarity determining residues GFTFTDYTMX (SEQ ID NO: 17), where X is preferably D or S; DVNPNSGGSIYNQRFKG (SEQ ID NO:18); and/or NLGPSFYFDY (SEQ ID NO:19), optionally comprising amino acid modifications of those CDR residues, e.g. where the modifications essentially maintain or improve affinity of the antibody. For example, an antibody variant for use in the methods of the present invention may have from about one to about seven or about five amino acid substitutions in the above variable heavy CDR sequences. Such antibody variants may be prepared by affinity maturation, e.g., as described below.

The humanized antibody may comprise variable light domain complementarity determining residues KASQDVSIGVA (SEQ ID NO:20); SASYX¹X²X³, where X¹ is preferably R or L, X² is preferably Y or E, and X³ is preferably T or S (SEQ ID NO:21); and/or QQYYIYPYT (SEQ ID NO:22), e.g. in addition to those variable heavy domain CDR residues in the preceding paragraph. Such humanized antibodies optionally comprise amino acid modifications of the above CDR residues, e.g. where the modifications essentially maintain or improve affinity of the antibody. For example, the antibody variant of interest may have from about one to about seven or about five amino acid substitutions in the above variable light CDR sequences. Such antibody variants may be prepared by affinity maturation, e.g., as described below.

The present application also contemplates affinity matured antibodies which bind HER2. The parent antibody may be a human antibody or a humanized antibody, e.g., one comprising the variable light and/or variable heavy sequences of SEQ ID Nos. 7 and 8, respectively (i.e. comprising the VL and/or VH of pertuzumab). An affinity matured variant of pertuzumab preferably binds to HER2 receptor with an affinity superior to that of murine 2C4 or pertuzumab (e.g. from about two or about four fold, to about 100 fold or about 1000 fold improved affinity, e.g. as assessed using a HER2-extracellular domain (ECD) ELISA). Exemplary variable heavy CDR residues for substitution include H28, H30, H34, H35, H64, H96, H99, or combinations of two or more (e.g. two, three, four, five, six, or seven of these residues). Examples of variable light CDR residues for alteration include L28, L50, L53, L56, L91, L92, L93, L94, L96, L97 or combinations of two or more (e.g. two to three, four, five or up to about ten of these residues).

Humanization of murine 4D5 antibody to generate humanized variants thereof, including trastuzumab, is described in U.S. Pat. Nos. 5,821,337, 6,054,297, 6,407,213, 6,639,055, 6,719,971, and 6,800,738, as well as Carter et al. PNAS (USA), 89:4285-4289 (1992). HuMAb4D5-8 (trastuzumab) bound HER2 antigen 3-fold more tightly than the mouse 4D5 antibody, and had secondary immune function (ADCC) which allowed for directed cytotoxic activity of the humanized antibody in the presence of human effector cells. HuMAb4D5-8 comprised variable light (V_{L}) CDR residues incorporated in a V_{L} K subgroup I consensus framework, and variable heavy (V_{H}) CDR residues incorporated into a V_{H} subgroup III consensus framework. The antibody further comprised framework region (FR) substitutions as positions: 71, 73, 78, and 93 of the V_{H} (Kabat numbering of FR residues; and a FR substitution at position 66 of the V_{L} (Kabat numbering of FR residues). trastuzumab comprises non-A allotype human γ1 Fc region.

Various forms of the humanized antibody or affinity matured antibody are contemplated. For example, the humanized antibody or affinity matured antibody may be an antibody fragment. Alternatively, the humanized antibody or affinity matured antibody may be an intact antibody, such as an intact IgG1 antibody.

(ii) Pertuzumab Compositions

In one embodiment of a HER2 antibody composition, the composition comprises a mixture of a main species pertuzumab antibody and one or more variants thereof. The preferred embodiment herein of a pertuzumab main species antibody is one comprising the variable light and variable heavy amino acid sequences in SEQ ID Nos. 5 and 6, and most preferably comprising a light chain amino acid sequence of SEQ ID No. 11, and a heavy chain amino acid sequence of SEQ ID No. 12 (including deamidated and/or oxidized variants of those sequences). In one embodiment, the composition comprises a mixture of the main species pertuzumab antibody and an amino acid sequence variant thereof comprising an amino-terminal leader extension. Preferably, the amino-terminal leader extension is on a light chain of the antibody variant (e.g. on one or two light chains of the antibody variant). The main species HER2 antibody or the antibody variant may be an full length antibody or antibody fragment (e.g. Fab of F(ab=)2 fragments), but preferably both are full length antibodies. The antibody variant herein may comprise an amino-terminal leader extension on any one or more of the heavy or light chains thereof. Preferably, the amino-terminal leader extension is on one or two light chains of the antibody. The amino-terminal leader extension preferably comprises or consists of VHS−. Presence of the amino-terminal leader extension in the composition can be detected by various analytical techniques including, but not limited to, N-terminal sequence analysis, assay for charge heterogeneity (for instance, cation exchange chromatography or capillary zone electrophoresis), mass spectrometry, etc. The amount of the antibody variant in the composition generally ranges from an amount that constitutes the detection limit of any assay (preferably N-terminal sequence analysis) used to detect the variant to an amount less than the amount of the main species antibody. Generally, about 20% or less (e.g. from about 1% to about 15%, for instance from 5% to about 15%) of the antibody molecules in the composition comprise an amino-terminal leader extension. Such percentage amounts are preferably determined using quantitative N-terminal sequence analysis or cation exchange analysis (preferably using a high-resolution, weak cation-exchange column, such as a PROPAC WCX-10™ cation exchange column). Aside from the amino-terminal leader extension variant, further amino acid sequence alterations of the main species antibody and/or variant are contemplated, including but not limited to an antibody comprising a C-terminal lysine residue on one or both heavy chains thereof, a deamidated antibody variant, etc.

Moreover, the main species antibody or variant may further comprise glycosylation variations, non-limiting examples of which include antibody comprising a G1 or G2 oligosaccharide structure attached to the Fc region thereof, antibody comprising a carbohydrate moiety attached to a light chain thereof (e.g. one or two carbohydrate moieties, such as glucose or galactose, attached to one or two light chains of the antibody, for instance attached to one or more lysine residues), antibody comprising one or two non-glycosylated heavy chains, or antibody comprising a sialidated oligosaccharide attached to one or two heavy chains thereof etc.

The composition may be recovered from a genetically engineered cell line, e.g. a Chinese Hamster Ovary (CHO) cell line expressing the HER2 antibody, or may be prepared by peptide synthesis.

For more information regarding exemplary pertuzumab compositions, see U.S. Pat. Nos. 7,560,111 and 7,879,325 as well as US 2009/0202546A1.

(iii) Trastuzumab Compositions

The trastuzumab composition generally comprises a mixture of a main species antibody (comprising light and heavy chain sequences of SEQ ID NOS: 13 and 14, respectively), and variant forms thereof, in particular acidic variants (including deamidated variants). Preferably, the amount of such acidic variants in the composition is less than about 25%, or less than about 20%, or less than about 15%. See, U.S. Pat. No. 6,339,142. See, also, Harris et al., J. Chromatography, B 752:233-245 (2001) concerning forms of trastuzumab resolvable by cation-exchange chromatography, including Peak A (Asn30 deamidated to Asp in both light chains); Peak B (Asn55 deamidated to isoAsp in one heavy chain); Peak 1 (Asn30 deamidated to Asp in one light chain); Peak 2 (Asn30 deamidated to Asp in one light chain, and Asp102 isomerized to isoAsp in one heavy chain); Peak 3 (main peak form, or main species antibody); Peak 4 (Asp102 isomerized to isoAsp in one heavy chain); and Peak C (Asp102 succinimide (Asu) in one heavy chain). Such variant forms and compositions are included in the invention herein.

(iv) Chemotherapy, Hormone Therapy, and G-CSF

5-fluorouracil, epirubicin, doxorubicin, cyclophosphamide, docetaxel, paclitaxel, G-CSF, and drugs suitable for adjuvant hormone therapy are commercially available and administered in accordance with local prescribing information and as described in the Examples.

### III. Selecting Patients for Therapy

Detection of HER2 can be used to select patients for treatment in accordance with the present invention. Several FDA-approved commercial assays are available to identify HER2-positive cancer patients. These methods include HERCEPTEST® (Dako) and PATHWAY® HER2 (immunohistochemistry (IHC) assays) and PathVysion® and HER2 FISH pharmDx™ (FISH assays). Users should refer to the package inserts of specific assay kits for information on the validation and performance of each assay.

For example, HER2 overexpression may be analyzed by IHC, e.g. using the HERCEPTEST® (Dako). Paraffin embedded tissue sections from a tumor biopsy may be subjected to the IHC assay and accorded a HER2 protein staining intensity criteria as follows:

Score 0 no staining is observed or membrane staining is observed in less than 10% of tumor cells.

Score 1+a faint/barely perceptible membrane staining is detected in more than 10% of the tumor cells. The cells are only stained in part of their membrane.

Score 2+a weak to moderate complete membrane staining is observed in more than 10% of the tumor cells.

Score 3+a moderate to strong complete membrane staining is observed in more than 10% of the tumor cells.

Those tumors with 0 or 1+ scores for HER2 overexpression assessment may be characterized as HER2-negative, whereas those tumors with 2+ or 3+ scores may be characterized as HER2-positive.

Tumors overexpressing HER2 may be rated by immunohistochemical scores corresponding to the number of copies of HER2 molecules expressed per cell, and can been determined biochemically:

0=0-10,000 copies/cell,

1+=at least about 200,000 copies/cell,

2+=at least about 500,000 copies/cell,

3+=at least about 2,000,000 copies/cell.

Overexpression of HER2 at the 3+ level, which leads to ligand-independent activation of the tyrosine kinase (Hudziak et al., Proc. Natl. Acad. Sci. USA, 84:7159-7163 (1987)), occurs in approximately 30% of breast cancers, and in these patients, relapse-free survival and overall survival are diminished (Slamon et al., Science, 244:707-712 (1989); Slamon et al., Science, 235:177-182 (1987)).

The presence of HER2 protein overexpression and gene amplification are highly correlated, therefore, alternatively, or additionally, the use of in situ hybridization (ISH), e.g. fluorescent in situ hybridization (FISH), assays to detect gene amplification may also be employed for selection of patients appropriate for treatment in accordance with the present invention. FISH assays such as the INFORM™ (sold by Ventana, Ariz.) or PathVysion® (Vysis, Ill.) may be carried out on formalin-fixed, paraffin-embedded tumor tissue to determine the extent (if any) of HER2 amplification in the tumor.

Most commonly, HER2-positive status is confirmed using archival paraffin-embedded tumor tissue, using any of the foregoing methods.

Preferably, HER2-positive patients having a 2+ or 3+ IHC score or who are FISH or ISH positive are selected for treatment in accordance with the present invention.

See also U.S. Pat. No. 7,981,418 for alternative assays for screening patients for therapy with pertuzumab, and the Examples.

### IV. Pharmaceutical Formulations

Therapeutic formulations of the HER2 antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), generally in the form of lyophilized formulations or aqueous solutions. Antibody crystals are also contemplated (see US Pat Appln 2002/0136719). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Lyophilized antibody formulations are described in WO 97/04801, expressly incorporated herein by reference.

Lyophilized antibody formulations are described in U.S. Pat. Nos. 6,267,958, 6,685,940 and 6,821,515, expressly incorporated herein by reference. The preferred HERCEPTIN® (trastuzumab) formulation is a sterile, white to pale yellow preservative-free lyophilized powder for intravenous (IV) administration, comprising 440 mg trastuzumab, 400 mg .alphaα,α-trehalose dehydrate, 9.9 mg L-histidine-HCl, 6.4 mg L-histidine, and 1.8 mg polysorbate 20, USP. Reconstitution of 20 mL of bacteriostatic water for injection (BWFI), containing 1.1% benzyl alcohol as a preservative, yields a multi-dose solution containing 21 mg/mL trastuzumab, at pH of approximately 6.0. For further details, see the trastuzumab prescribing information.

The preferred pertuzumab formulation for therapeutic use comprises 30 mg/mL pertuzumab in 20 mM histidine acetate, 120 mM sucrose, 0.02% polysorbate 20, at pH 6.0. An alternate pertuzumab formulation comprises 25 mg/mL pertuzumab, 10 mM histidine-HCl buffer, 240 mM sucrose, 0.02% polysorbate 20, pH 6.0.

The formulation of the placebo used in the clinical trials described in the Examples is equivalent to pertuzumab, without the active agent.

The formulation herein may also contain more than one active compound as necessary for the particular indication being treated, preferably those with complementary activities that do not adversely affect each other. Various drugs which can be combined with the HER dimerization inhibitor are described in the Method Section below. Such molecules are suitably present in combination in amounts that are effective for the purpose intended.

The formulations to be used for in vivo administration must be sterile. This is readily accomplished by filtration through sterile filtration membranes.

### V. Treatment Methods

The invention concerns a method for the treatment of breast cancer comprising neoadjuvant administration to a patient with HER2-positive locally advanced, inflammatory, or early-stage breast cancer of an effective amount of a combination of pertuzumab and trastuzumab following anthracycline-based chemotherapy, wherein the combined administration of pertuzumab and trastuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.

Pertuzumab and trastuzumab are administered according to Prescribing Information and as described throughout the disclosure, including but not limited to the examples herein.

In one embodiment, dose-dense doxorubicin and cyclophosphamide (ddAC) is given every 2 weeks, followed by weekly administration of a taxane, e.g. paclitaxel, and combined administration of pertuzumab and trastuzumab every three weeks. In a particular embodiment, ddAC is given every two weeks for four cycles with granulocyte colony-stimulating factor (G-CSF) support as needed, followed by weekly paclitaxel administration for 12 weeks, with pertuzumab and trastuzumab given every three weeks from the start of paclitaxel.

In another embodiment, 5-fluorouracil, epirubicin, and cyclophosphamide (FEC) is given every three weeks for four cycles, followed by docetaxel given every three weeks for four cycles, with pertuzumab and trastuzumab given every 3 weeks from the start of docetaxel.

In all embodiments, the patients may receive at least four cycles of neoadjuvant pertuzumab+trastuzumab, and more than four cycles of neoadjuvant pertuzumab+trastuzumab administration are specifically included. Thus, in various embodiments, 5 cycles, or 6 cycles, or 7 cycles, or 8 cycles of neoadjuvant pertuzumab+trastuzumab may be administered. Administration of at least four cycles of neoadjuvant pertuzumab+trastuzumab is beneficial in increasing the tpCR rate, especially following administration of ddAC.

Dosages and schedules for chemotherapy used to treat HER2-positive breast cancer are disclosed in the examples below, but other dosages and schedules are known and contemplated according to the invention herein.

### VI. Articles of Manufacture

In another embodiment of the invention, an article of manufacture containing materials useful for the treatment of breast cancer is provided. The article of manufacture comprises a vial with a fixed dose of the HER2 (pertuzumab), wherein the fixed dose is approximately 420 mg, approximately 525 mg, approximately 840 mg, or approximately 1050 mg of the HER antibody.

The article of manufacture preferably further comprises a package insert. The package insert may provide instructions to administer the fixed dose to a breast cancer patient,

In one embodiment, the article of manufacture comprises two vials, wherein a first vial contains a fixed dose of approximately 840 mg of pertuzumab, and a second vial contains a fixed dose of approximately 420 mg of pertuzumab.

In another embodiment, the article of manufacture of comprises two vials, wherein a first vial contains a fixed dose of approximately 1050 mg of pertuzumab, and a second vial contains a fixed dose of approximately 525 mg of pertuzumab.

In one embodiment of an article of manufacture herein comprises an intravenous (IV) bag containing a stable mixture of pertuzumab and trastuzumab suitable for administration to a cancer patient. Optionally, the mixture is in saline solution; for example comprising about 0.9% NaCl or about 0.45% NaCl. An exemplary IV bag is a polyolefin or polyvinyl chloride infusion bag, e.g. a 250 mL IV bag. According to one embodiment of the invention, the mixture includes about 420 mg or about 840 mg of pertuzumab and from about 200 mg to about 1000 mg of trastuzumab (e.g. from about 400 mg to about 900 mg of trastuzumab).

Optionally, the mixture in the IV bag is stable for up to 24 hours at 5° C. or 30° C. Stability of the mixture can be evaluated by one or more assays selected from the group consisting of: color, appearance and clarity (CAC), concentration and turbidity analysis, particulate analysis, size exclusion chromatography (SEC), ion-exchange chromatography (IEC), capillary zone electrophoresis (CZE), image capillary isoelectric focusing (iCIEF), and potency assay.

In one embodiment the article of manufacture comprises a vial with pertuzumab and a package insert, wherein the package insert provides at least part of the safety data shown in FIGS. 10-15.

In another embodiment, the article of manufacture comprises a single-dose vial containing about 420 mg of pertuzumab.

The invention also concerns a method for making an article of manufacture comprising packaging together a vial with pertuzumab therein and a package insert, wherein the package insert provides the safety data shown in FIGS. 10-15.

### VII. Deposit of Biological Materials

The following hybridoma cell lines have been deposited with the American Type Culture

Collection, 10801 University Boulevard, Manassas, Va. 20110-2209, USA (ATCC):

| Antibody Designation | ATCC No. | Deposit Date |
|---|---|---|
| 4D5 | ATCC CRL 10463 | May 24, 1990 |
| 2C4 | ATCC HB-12697 | Apr. 8, 1999 |

TABLE OF SEQUENCES

| Description | SEQ ID NO | FIG. |
|---|---|---|
| HER2 domain I | 1 | 1 |
| HER2 domain II | 2 | 1 |
| HER2 domain III | 3 | 1 |
| HER2 domain IV | 4 | 1 |
| 2C4 variable light | 5 | 2A |
| 2C4 variable heavy | 6 | 2B |
| 574/pertuzumab variable light | 7 | 2A |
| 574/pertuzumab variable heavy | 8 | 2B |
| human V_{L} consensus framework | 9 | 2A |
| Human V_{H} consensus framework | 10 | 2B |
| pertuzumab light chain | 11 | 3A |
| pertuzumab heavy chain | 12 | 3B |
| trastuzumab light chain | 13 | 4A |
| trastuzumab heavy chain | 14 | 4B |
| Variant pertuzumab light chain | 15 | 5A |
| Variant pertuzumab heavy chain | 16 | 5B |
| GFTFTDYTMX | 17 | |
| DVNPNSGGSIYNQRFKG | 18 | |
| NLGPSFYFDY | 19 | |
| KASQDVSIGVA | 20 | |
| SASYX¹X²X³ | 21 | |
| QQYYIYPYT | 22 | |

Further details of the invention are illustrated by the following non-limiting Examples. The disclosures of all citations in the specification are expressly incorporated herein by reference.

A list of abbreviations and definition of terms, as used throughout the specification, including the Examples, is provided in the following table.

| Abbreviation | Definition |
|---|---|
| AC | doxorubicin (Adriamycin ®) plus cyclophosphamide |
| ADCC | antibody-dependent cell-mediated cytotoxicity |
| AE | adverse event |
| ARDS | acute respiratory distress syndrome |
| ATA | anti-therapeutic antibody |
| BCS | breast-conserving surgery |
| bpCR | breast pathologic complete response |
| BSA | body surface area |
| CALGB | Cancer and Leukemia Group B |
| CBE | clinical breast examination |
| CHF | congestive heart failure |
| CISH | chromogenic in situ hybridization |
| CR | complete response |
| CSR | Clinical Study Report |
| CT | computed tomography |
| CTCAE | Common Terminology Criteria for Adverse Events |
| D | Docetaxel |
| DCarbH | docetaxel, carboplatin, and trastuzumab (Herceptin ®) |
| | (also known as TCH) |
| DCIS | ductal carcinoma in situ |
| dd | dose-dense |
| ddAC | dose-dense doxorubicin (Adriamycin ®) plus |
| | cyclophosphamide |
| DFS | disease-free survival |
| EBC | early breast cancer |
| EBCTCG | Early Breast Cancer Trialists' Collaborative Group |
| ECG | electrocardiogram |
| ECHO | echocardiogram |
| ECOG | Eastern Cooperative Oncology Group |
| eCRF | electronic Case Report Form |
| EDC | electronic data capture |
| EFS | event-free survival |
| EGFR | epidermal growth factor receptor |
| ER | estrogen receptor |
| ESMO | European Society for Medical Oncology |
| FFPE | formalin-fixed paraffin-embedded |
| FISH | fluorescent in situ hybridization |
| GCG | German Breast Group |
| G-CSF | granulocyte colony-stimulating factor |
| H | Herceptin |
| HER2 | human epidermal growth factor receptor 2 |
| HR | hazard ratio |
| IB | Investigator's Brochure |
| IBC | inflammatory breast cancer |
| ICH | International Conference on Harmonisation |
| iDFS | invasive disease-free survival |
| IMP | investigational medicinal product |
| IND | investigational new drug |
| ISH | in situ hybridization |
| ITT | intent-to-treat |
| IV | Intravenous |
| IUD | intrauterine device |
| IxRS | interactive voice/web response system |
| LABC | locally advanced breast cancer |
| LCIS | lobular carcinoma in situ |
| LPLV | last patient, last visit |
| LVEF | left ventricular ejection fraction |
| LVSD | left ventricular systolic dysfunction |
| MAPK | mitogen-activated protein kinase |
| MBC | metastatic breast cancer |
| MRI | magnetic resonance imaging |
| mRNA | messenger RNA |
| MUGA | multiple-gated acquisition scan |
| NCCN | National Comprehensive Cancer Network |
| NCCTG | North Central Cancer Treatment Group |
| NCI | National Cancer Institute |
| NSABP | National Surgical Adjuvant Breast and Bowel Project |
| NYHA | New York Heart Association |
| OS | overall survival |
| P | Paclitaxel |
| pCR | pathological complete response |
| PET | positron emission tomography |
| PFS | progression-free survival |
| PgR | progesterone receptor |
| PH | Perjeta ® and Herceptin ® |
| PI3K | phosphoinositol 3-kinase |
| Pla | Placebo |
| PR | partial response |
| PVC | polyvinyl chloride |
| RCB | Residual Cancer Burden |
| RCR | Roche Clinical Repository |
| RECIST | Response Evaluation Criteria in Solid Tumors |
| RT | radiotherapy |
| SD | stable disease |
| SISH | silver in situ hybridization |
| SLN | sentinel lymph node |
| SLNB | sentinel lymph node biopsy |
| SWFI | sterile water for injection |
| T | paclitaxel (Taxol ®) |
| TCH | docetaxel (Taxotere ®), cyclophosphamide, and |
| | trastuzumab (Herceptin ®) (abbreviated to DCarbH in |
| | this document) |
| TH | paclitaxel plus Herceptin ® |
| tpCR | total pathologic complete response |
| ULN | upper limit of normal |

### Example 1

### Phase II Clinical Study to Evaluate Neoadjuvant Administration of PERJETA® in Combination with HERCEPTIN® and Standard Neoadjuvant Anthracycline-Based Chemotherapy

### Study Design

### Description of Study

Overview of Study Design

This is a non-randomized, open-label, multicenter, multinational, Phase II trial including two parallel groups of patients. A total of approximately 400 patients are planned: approximately 200 patients in each treatment cohort. Patients considered suitable for neoadjuvant treatment with HERCEPTIN® plus anthracycline/taxane-based chemotherapy will be allocated to receive one of the two following regimens:
Cohort A (ddAC-*T+PH): Dose-dense doxorubicin and cyclophosphamide given every 2 weeks for four cycles with G-CSF support as needed according to local guidelines, followed by weekly paclitaxel for 12 weeks, with PERJETA® and HERCEPTIN® every 3 weeks from the start of paclitaxel (four cycles of PERJETA® and HERCEPTIN® prior to surgery). OR
Cohort B (FEC-*D+PH): 5-fluorouracil, epirubicin and cyclophosphamide given every 3 weeks for four cycles, followed by docetaxel given every 3 weeks for four cycles. In addition, patients will receive PERJETA® and HERCEPTIN® every 3 weeks from the start of docetaxel (four cycles of PERJETA® and HERCEPTIN® prior to surgery).

The choice of neoadjuvant treatment regimen will be made by the investigator on an investigator-specific basis (i.e., only one cohort will be opened at a time at any given site; the investigator may not enroll patients into both cohorts simultaneously). If an investigator requests to change cohorts during the enrollment period of the study, the study team will review the circumstances before approving the request.

Following surgery, patients in both treatment cohorts will receive further adjuvant PERJETA® and HERCEPTIN® every 3 weeks (13 cycles), such that a total of 17 cycles of PERJETA® and HERCEPTIN® therapy are given during the study. Radiotherapy and adjuvant hormonal therapy will also be given as clinically indicated according to applicable guidelines.

The study is primarily intended to assess the cardiac safety of two commonly used neoadjuvant anthracycline/taxane-based chemotherapy regimens when given in combination with neoadjuvant PERJETA® and HERCEPTIN®. General safety and efficacy (notably the pCR rate) will also be assessed.

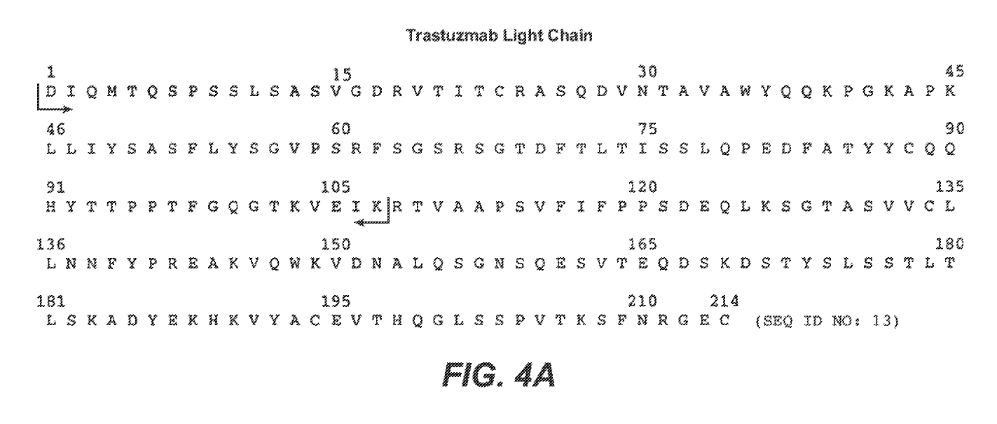

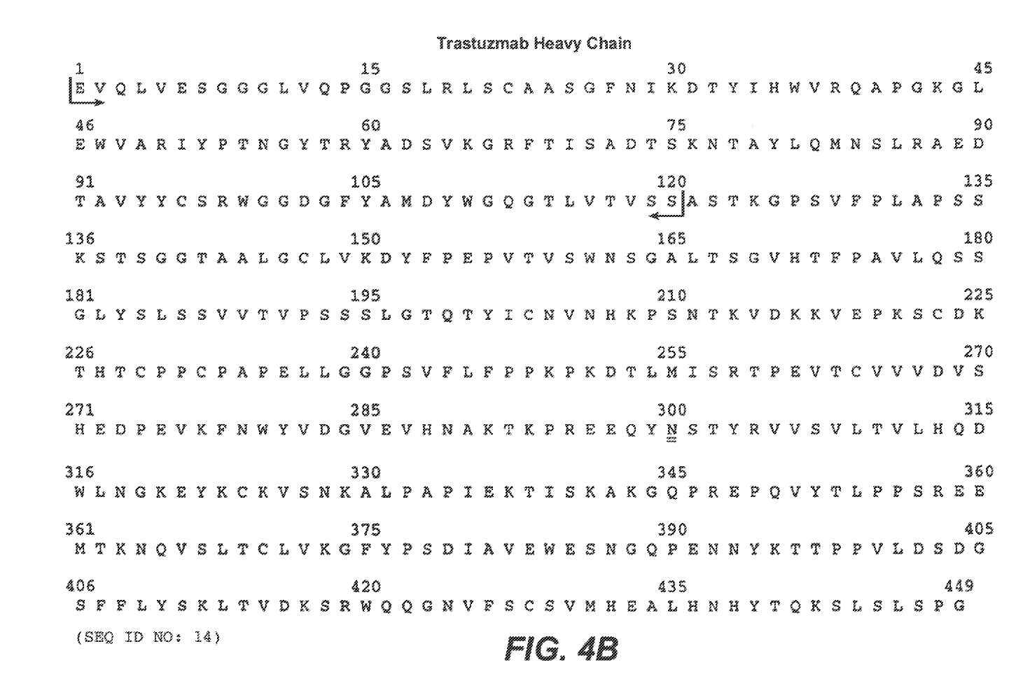

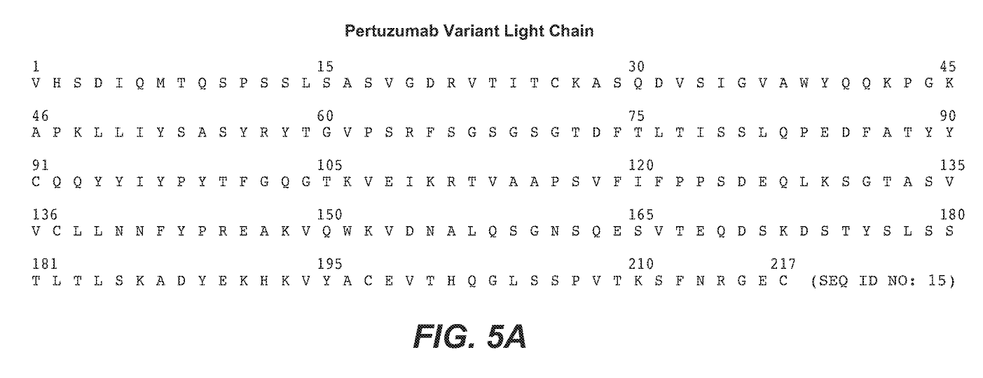

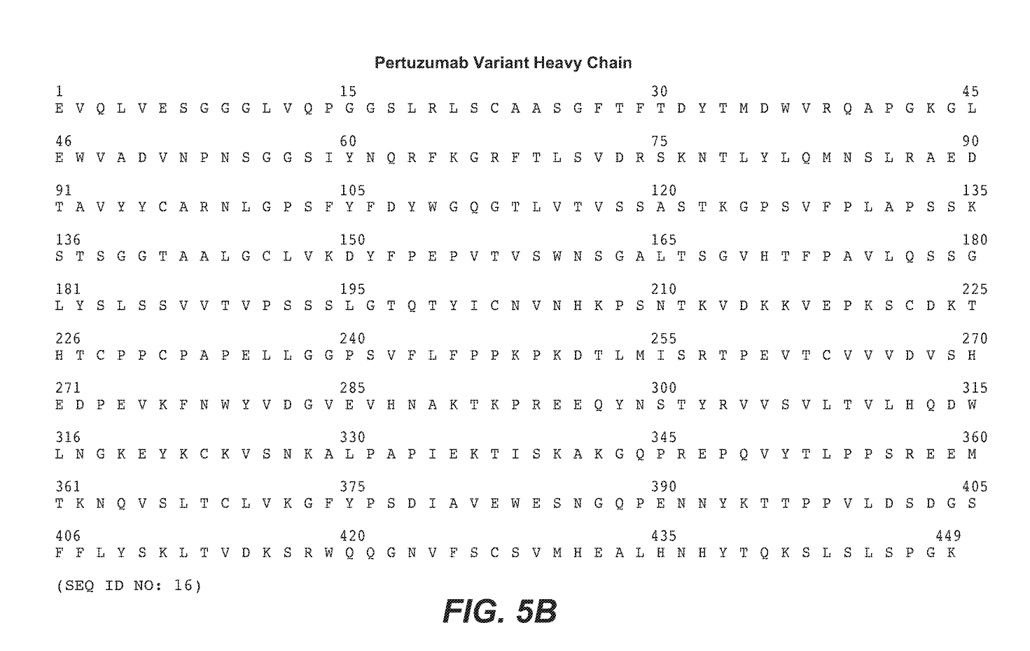

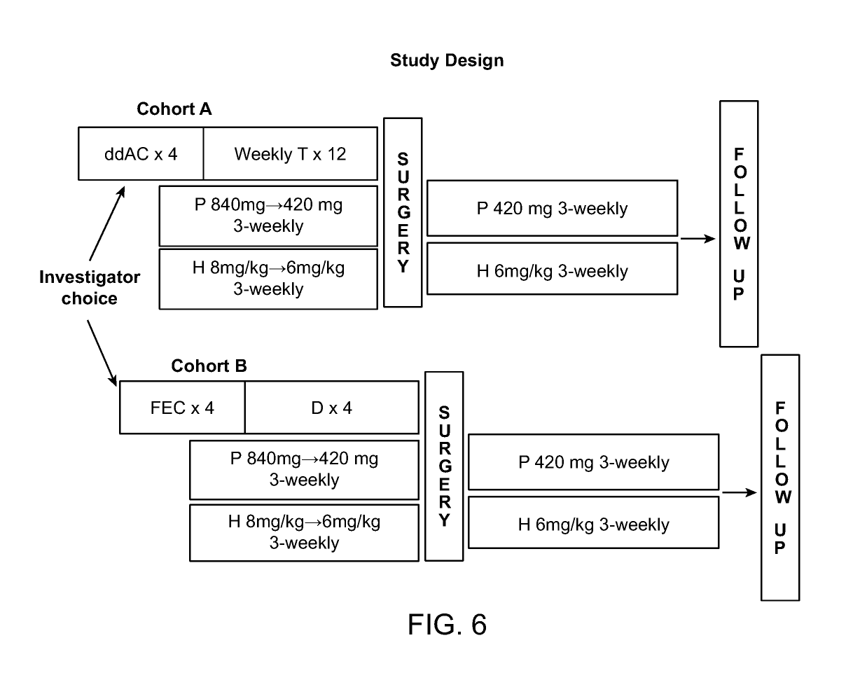

The study design is illustrated in FIG. 4. Note: For Cohort A, the doses of ddAC are doxorubicin 60 mg/m² and cyclophosphamide 600 mg/m². ddAC is given every 2 weeks. The dose of paclitaxel is 80 mg/m². For Cohort B, the doses of FEC are 5-fluorouracil 500 mg/m², epirubicin 100 mg/m², and cyclophosphamide 600 mg/m². FEC is given every 3 weeks. The starting dose of docetaxel is 75 mg/m² in Cycle 5 (the first docetaxel cycle), then 100 mg/m² for Cycles 6-8, if no dose-limiting toxicity occurs. D is given every 3 weeks.

End of Study

The end of the study will be 5 years after enrollment of the last patient in the study (or when all patients have died or the trial is terminated by the Sponsor, whichever is earliest). This data point will be considered last patient, last visit (LPLV). The study is expected to last approximately 6.5 years, assuming a recruitment period of approximately 1.5 years, time on treatment for each patient of approximately 1 year (including the neoadjuvant and adjuvant treatment periods), and follow-up for cardiac safety and efficacy for a further 4 years.

Rationale for Test Product Dosage

The chemotherapy regimens used in this study are based on published data and routine clinical usage, as well as established clinical practice guidelines (e.g., NCCN guidelines). The doses of chemotherapy, PERJETA®, and HERCEPTIN® are all consistent with the prescribing information for each agent. 5-fluorouracil, epirubicin, doxorubicin, cyclophosphamide, docetaxel, paclitaxel, and G-CSF are administered in accordance with local prescribing information, and these drugs are not regarded as investigational medicinal products (IMPs). PERJETA® and HERCEPTIN® are considered IMPs in this study.

All chemotherapy and antibody treatments are given intravenously.

In both cohorts, PERJETA® is given as an 840-mg intravenous (IV) loading dose, followed by 420 mg IV every 3 weeks; HERCEPTIN® is given as an 8 mg/kg IV loading dose, followed by 6 mg/kg IV every 3 weeks. After surgery, patients continue to receive PERJETA® and HERCEPTIN® in the adjuvant setting (13 cycles) until a total of 17 cycles of PERJETA® and HERCEPTIN® have been administered in the study.

Cohort A (ddAC→T+PH)

In Cohort A (ddAC→*T+PH), the doses and schedule of chemotherapy are based on routine practice and NCCN guidelines, as follows: doxorubicin 60 mg/m² and cyclophosphamide 600 mg/m² given every 2 weeks for four cycles, followed (2 weeks later) by weekly paclitaxel 80 mg/m² for 12 weeks. PERJETA® and HERCEPTIN® are given every 3 weeks from the start of paclitaxel so that a total of four cycles of PERJETA® and HERCEPTIN® are given during the neoadjuvant period. During ddAC, patients should receive G-CSF support according to local practice guidelines.

Cohort B (FEC→D+PH)

The dose and schedule of chemotherapy in Cohort B (FEC→*D+PH) are based on those evaluated in Arm B of the TRYPHAENA study (5-fluorouracil 500 mg/m², epirubicin 100 mg/m², cyclophosphamide 600 mg/m², given every 3 weeks; followed by docetaxel 75 mg/m², increasing to 100 mg/m² if tolerated, given every 3 weeks). The doses are consistent with doses given in routine practice and the regimen was well tolerated when given in combination with PERJETA® and HERCEPTIN®.

Outcome Measures

Safety Outcome Measures

The safety outcome measures for this study are as follows:
Incidence and severity of cardiac events, as assessed by the investigator using NCI CTCAE v4.0 (and NYHA for symptomatic LVSD)
Changes in LVEF over the course of the study assessed using local ECHO or MUGA scans. Whenever possible, patients will be assessed throughout the study by the same assessor and with the same technique used at baseline. A clinically significant LVEF decline is defined as a decline of ≥10%-points from baseline and to a value of <50%.
Incidence and severity of other adverse events and serious adverse events
Laboratory test abnormalities
Incidence of anti-therapeutic antibodies (ATAs) to PERJETA® and their relationship to safety events

All patients who receive at least one dose of any component of study treatment will be included in the safety analysis population. The primary cardiac safety evaluation will occur after all patients have completed neoadjuvant therapy (or have withdrawn from the study or are lost to follow-up). At this time, the main cardiac safety parameters of interest are as follows:
Incidence of NYHA Class III and IV heart failure during the neoadjuvant period, as assessed by the investigator. On the basis of the observed rates of Grade ≥3 LVSD (approximately equivalent to NHYA Class III/W heart failure) in the NEOSPHERE and TRYPHAENA studies, in which neoadjuvant PERJETA® and HERCEPTIN® were given with chemotherapy for up to six cycles, the incidence of NYHA Class III and IV heart failure during neoadjuvant treatment is estimated to be <3%.
Incidence of clinically significant LVEF declines (≥10%-points from baseline and to a value of <50%) during the neoadjuvant period. On the basis of the observed rates in the NEOSPHERE and TRYPHAENA studies, the underlying incidence of clinically significant LVEF declines during neoadjuvant treatment is estimated to be ≤6%.

Cardiac safety will continue to be assessed in all patients throughout the adjuvant and post-treatment period. Additional analyses of these parameters (and other safety and efficacy data) will be conducted after the primary analysis at the following time points:
After all patients have completed adjuvant anti-HER2 therapy (or have withdrawn from the study or are lost to follow-up)
At the end of the study (5 years after the last patient was enrolled)

Efficacy Outcome Measures

Efficacy will be assessed at the time of the primary analysis and at other key time points. The efficacy outcome measures for this study will be assessed in the intent-to-treat (ITT) population (all patients enrolled) and are as follows:
tpCR, defined as eradication of invasive disease in the breast and axilla (ypT0/is ypN0), according to the local pathologist's assessment. Pathologic response to therapy is defined at the time of surgery, and the tpCR rate is the proportion of patients in the ITT population who achieve a tpCR.
Clinical response, defined as complete response (CR), partial response (PR), stable disease (SD), or progressive disease (PD) prior to surgery. The clinical response rate is defined as the proportion of patients in the ITT population who achieve a CR or PR prior to surgery. Tumor response will be assessed prior to each new cycle of therapy by clinical examination, mammography, and/or other methods of evaluation as per local practice. Response will be assessed by the investigator as per local practice based on the principles of Response Evaluation Criteria in Solid Tumors (RECIST) Version 1.1 (Eisenhauer et al., Eur J Cancer 2009; 45:228-47).
EFS, defined as the time from enrollment to the first occurrence of PD or relapse, as determined by the investigator, or death from any cause. Ipsilateral or contralateral in situ disease and second primary non-breast cancers (including in situ carcinomas and non-melanoma skin cancers) will not be counted as PD or relapse.
iDFS, defined as the time from the first date of no disease (i.e., the date of primary surgery) to the first documentation of progressive invasive disease, relapse, or death from any cause. Ipsilateral or contralateral in situ disease and second primary non-breast cancers (including in situ carcinomas and non-melanoma skin cancers) will not be counted as PD or relapse.
OS, defined as the time from enrollment to death from any cause.

Exploratory Outcome Measures

The exploratory outcome measures for this study are as follows:
bpCR, defined as eradication of invasive disease in the breast (ypT0/is)
GBG pCR, defined as eradication of invasive and in situ disease in the breast, and invasive disease in the axilla (ypT0 ypN0)
BCS, defined as quadrantectomy or lumpectomy. The BCS rate will be provided for all female patients in the ITT population (i.e., described as the overall BCS rate), for female patients with T2 or T3 tumors at study entry, and for female patients with T2 or T3 tumors for whom mastectomy was planned at study entry.
Re-excision surgery, defined as surgery on a separate occasion (i.e., requiring a separate anesthetic) following initial BCS to remove residual tumor
Gene expression, as determined by messenger RNA (mRNA) expression levels measured on a multiplex platform. Gene panels (e.g., PAM50 panel) and single genes will be assessed. A panel of genes relevant to breast cancer will be assessed on a multiplex platform. Intrinsic breast cancer subtypes will be identified by applying the PAM50 gene set, as described by Parker et al. (2009). In addition to the PAM50 classifier, other gene signatures may be evaluated; e.g., those signifying an activated PI3K pathway or related to immune response. The relationship between the intrinsic breast cancer subtypes (notably the luminal B and HER2-enriched subtypes included in this study) and outcome (defined by pCR) are of particular interest. Other molecular profiles or single markers will be evaluated as appropriate and as sample size allows.

Investigational Medicinal Products

PERJETA® and HERCEPTIN® are both investigational medicinal products for this study.

Test Product

Pertuzumab (PERJETA) is given as 840 mg intravenous (IV) loading dose, then 420 mg IV every 3 weeks.

Dosing Regimen

In both cohorts, trastuzumab (HERCEPTIN) is given as an 8 mg/kg IV loading dose, then 6 mg/kg IV every 3 weeks.
Cohort A: Doxorubicin 60 mg/m² IV and cyclophosphamide 600 mg/m² IV administered every 2 weeks (ddAC) for four cycles (Cycles 1-4), followed (2 weeks later) by paclitaxel 80 mg/m² IV weekly for 12 weeks (Cycles 5-8), with PERJETA® and HERCEPTIN® every 3 weeks during Cycles 5-8 (from the start of paclitaxel; four cycles of PERJETA® and HERCEPTIN® in total during the neoadjuvant period). During ddAC, patients should receive G-CSF support according to local practice guidelines. After surgery, patients will continue to receive PERJETA® and HERCEPTIN® in the adjuvant setting until a total of 17 cycles of PERJETA® and HERCEPTIN® have been given.
Cohort B: 5-fluorouracil 500 mg/m² IV, epirubicin 100 mg/m² IV, and cyclophosphamide 600 mg/m² IV every 3 weeks for four cycles (Cycles 1-4), followed (3 weeks later) by docetaxel (75 mg/m² for the first dose, then 100 mg/m² at subsequent doses if no dose-limiting toxicity occurs) every 3 weeks for four cycles (Cycles 5-8), with PERJETA® and HERCEPTIN® every 3 weeks during Cycles 5-8 (from the start of docetaxel; four cycles of PERJETA® and HERCEPTIN® in total during the neoadjuvant period). After surgery, patients should continue to receive PERJETA® and HERCEPTIN® in the adjuvant setting until a total of 17 cycles of PERJETA® and HERCEPTIN® have been given.

All treatment doses should be based on actual body weight and not ideal body weight. If a patient's body weight increases or decreases by ≥10% from baseline during the course of treatment, the body surface area and dose of chemotherapy and/or HERCEPTIN® should be recalculated.

Non-Investigational Medicinal Products

5-fluorouracil, epirubicin, doxorubicin, cyclophosphamide, docetaxel, paclitaxel, and G-CSF are administered in accordance with local prescribing information, so these drugs are not regarded as Investigational Medicinal Products.

After surgery, patients with hormone receptor-positive disease should receive adjuvant hormone therapy according to guidelines included in the protocol. Postoperative radiotherapy is also indicated according to guidelines included in the protocol.

All patients will receive full supportive care, including anti-emetics (e.g., serotonin antagonists, benzodiazepines), anti-diarrheal agents (e.g., loperamide), short-term corticosteroids to treat or prevent allergic or infusion reactions, H1 and H2 antagonists (e.g., diphenhydramine, cimetidine), analgesics (e.g., paracetamol/acetaminophen, meperidine, opioids), and antibiotics as clinically indicated.

Statistical Methods

Primary Analysis

Efficacy analyses will be performed on the ITT population, defined as all patients enrolled in each treatment cohort. Patients will be analyzed according to the treatment cohort into which they were enrolled.

Safety analyses will be performed on the safety population. Enrolled patients who fail to receive any component of their planned study medication (i.e., who receive no neoadjuvant chemotherapy, PERJETA®, or HERCEPTIN® at all) will be excluded from the safety population. Patients will be analyzed for safety according to the treatment cohort into which they were enrolled because the choice of treatment regimen is made by the investigator.

No comparisons will be made between the efficacy and safety results of the two treatment cohorts. All analyses will be descriptive.

Both efficacy and safety data will be analyzed at the following time points:
After all patients have completed neoadjuvant therapy (or have withdrawn from the study or are lost to follow-up); this is the primary analysis time point.
After all patients have completed adjuvant therapy (or have withdrawn from the study or are lost to follow-up)
At the end of the study (5 years after the last patient is enrolled)

However, only limited efficacy analyses (primarily pCR) are planned for the first two time points because iDFS, EFS, and OS data will be relatively immature at this stage of the study.

Results of the primary analysis (after the completion of neoadjuvant therapy) will be included in the primary clinical study report (CSR). Results of subsequent analyses will be included in update CSRs.

Materials and Methods

Patients

The target population is adult men and women with locally advanced, inflammatory, or early-stage HER2-positive breast cancer (with primary tumors >2 cm in diameter or node-positive disease) scheduled to receive neoadjuvant therapy.

Inclusion Criteria

Patients must meet the following criteria for study entry:
Male and female patients with locally advanced, inflammatory, or early-stage, unilateral, and histologically confirmed invasive breast cancer. Patients with inflammatory breast cancer must be able to have a core needle biopsy.
Primary tumor >2 cm in diameter, or >5 mm in diameter and node-positive (clinically, on imaging, or on cytology/histopathology)
HER2-positive breast cancer confirmed by a central laboratory (3+ by immunohistochemistry or HER2 amplification by in situ hybridization with a ratio of HER2 gene signals to centromere 17 signals ≥2.0)
Availability of formalin-fixed, paraffin-embedded (FFPE) tumor tissue block for central confirmation of HER2, hormone receptor status, and molecular subtyping
Able and willing to provide written informed consent and to comply with the study protocol
Age ≥18 years
Baseline LVEF ≥55% (measured by ECHO or MUGA)
Eastern Cooperative Oncology Group (ECOG) performance status ≤1
At least 4 weeks since major unrelated surgery, with full recovery
A negative serum pregnancy test must be available for premenopausal women and for women <12 months after the onset of menopause, unless they have undergone surgical sterilization.
Women of childbearing potential and male participants with partners of childbearing potential must agree to use a “highly effective,” non-hormonal form of contraception or two “effective” forms of non-hormonal contraception by the patient and/or partner. Contraception must continue for the duration of study treatment and for at least 7 months after the last dose of study treatment.
Metastatic disease (Stage IV) or bilateral breast cancer
Patients who have had an incisional biopsy of the primary tumor or the primary tumor excised
Prior breast or non-breast malignancy within 5 years prior to study entry, except for carcinoma in situ and basal cell and squamous cell carcinoma of the skin. Patients with malignancies occurring more than 5 years prior to study entry are permitted if curatively treated.
Any previous systemic therapy (including chemotherapy, immunotherapy, HER2-targeted agents, and antitumor vaccines) for cancer, or radiation therapy for cancer
Patients with a past history of ductal carcinoma in situ (DCIS) or lobular carcinoma in situ (LCIS) are not allowed to enter the study if they have received any systemic therapy for its treatment or radiation therapy to the ipsilateral breast (they are allowed to enter the study if treated with surgery alone).
High-risk patients who have received chemopreventative drugs in the past are not allowed to enter the study.
Inadequate bone marrow function (e.g., absolute neutrophil count <1.5×10⁹/L, platelet count <100×10⁹/L, and hemoglobin <9 g/dL)
Impaired liver function (e.g., serum [total] bilirubin >1.25× upper limit of normal [ULN] [with the exception of Gilbert's syndrome], AST and ALT>1.25×ULN, albumin <25 g/L)
Inadequate renal function with serum creatinine >1.5×ULN
Poorly controlled hypertension (e.g., systolic blood pressure >180 mm Hg and/or diastolic blood pressure >100 mm Hg), angina requiring anti-anginal medication, history of CHF of any NYHA classification, serious or uncontrolled cardiac arrhythmia requiring treatment (exceptions: controlled atrial fibrillation with heart rate ≤100 bpm at rest, and paroxysmal supraventricular tachycardia), history of myocardial infarction within 6 months of enrollment, or LVEF<55%
Dyspnea at rest or other diseases that require continuous oxygen therapy
Severe, uncontrolled systemic disease
Patients with poorly controlled diabetes or with evidence of clinically significant diabetic vascular complications
Pregnant or lactating women
Patients who received any investigational treatment within 4 weeks of study start
Patients with known infection with HIV, hepatitis B virus, or hepatitis C virus
Current chronic daily treatment with corticosteroids (dose >10 mg methylprednisolone or equivalent [excluding inhaled steroids])
Known hypersensitivity to any of the study drugs or excipients
Patients assessed by the investigator to be unable or unwilling to comply with the requirements of the protocol.

Study Treatment

Study treatment is defined as neoadjuvant (pre-surgery) and adjuvant (post-surgery) treatment. Throughout the study, the IMPs are PERJETA® and HERCEPTIN®.

The choice of neoadjuvant treatment regimen will be made by the investigator on an investigator-specific basis (i.e., only one cohort will be opened at a time at any given site; the investigator may not enroll patients into both cohorts simultaneously). If an investigator requests to change cohorts during the enrollment period of the study, the study team will review the circumstances before approving the request.

Prescribing of hormone therapy, where applicable, is in accordance with guidelines provided. Radiotherapy is also given as clinically indicated in accordance with guidelines provided. Details of hormone therapy and radiotherapy will be recorded on the electronic Case Report Form (eCRF).

Formulation, Packaging, and Handling

Formulation of PERJETA®

Perjeta® is provided as a single-use formulation containing 30 mg/mL pertuzumab formulated in 20 mM L-histidine (pH 6.0), 120 mM sucrose, and 0.02% polysorbate-20. Each 20-cc vial contains approximately 420 mg of pertuzumab (14.0 mL/vial).

For further details, refer to the PERJETA® IB or local prescribing information for PERJETA®.

Labeling of PERJETA®

PERJETA® will be labeled according to the regulatory requirements in each country, as well as in accordance with International Conference of Harmonisation (ICH) Good Clinical Practice. The study Sponsor will provide PERJETA® to all study sites labeled for investigational use only.

Storage of PERJETA0

Vials of PERJETA® are shipped at a temperature ranging from 2° C.-8° C. (36° F.-46° F.), and must be placed in a refrigerator (same temperature range) immediately upon receipt to ensure optimal retention of physical and biochemical integrity, and should remain refrigerated until immediately prior to use. Temperature logs must be maintained (in accordance with local pharmacy practice) on the refrigerator to ensure proper storage conditions. If a temperature deviation from the allowed 2° C.-8° C. is found either during shipment or storage, contact the Sponsor to determine if the drug is still appropriate for use.

The PERJETA® vials may not be shaken. All vials should be stored within the outer carton and protected from light. The medication must not be used beyond the use by date information provided on the IMP kit label.

Preparation of PERJETA®

Because the PERJETA® formulation does not contain a preservative, the vial seal may only be punctured once. Any remaining solution should be discarded.

The indicated volume of PERJETA® solution should be withdrawn from the vials and added to a 250-cc IV bag of 0.9% sodium chloride injection. The bag should be gently inverted to mix the solution, but should not be shaken vigorously. The solution should be visually inspected for particulates and discoloration prior to administration. The entire volume within the bag should be administered as a continuous IV infusion. The volume contained in the administration tubing should be completely flushed using a 0.9% sodium chloride injection.

The solution of PERJETA® for infusion, diluted in polyvinyl chloride (PVC) or non-PVC polyolefin bags containing 0.9% sodium chloride injection, may be stored at 2° C.-8° C. (36° F.-46° F.) for up to 24 hours prior to use. Diluted PERJETA® has been shown to be stable for up to 24 hours at room temperature (2° C.-25° C.). However, because diluted Perjeta contains no preservative, the aseptically diluted solution should be stored refrigerated (2° C.-8° C.) for no more than 24 hours.

A rate-regulating device may be used for all study-drug infusions. When the study drug IV bag is empty, 50 mL of 0.9% sodium chloride injection may be added to the IV bag or an additional bag may be hung, and the infusion may be continued for a volume equal to that of the tubing to ensure complete delivery of the study drug.

If extravasation of the study drug infusion occurs, the following steps should be taken:
Discontinue the infusion.
Treat the extravasation according to institutional guidelines for extravasation of a non-caustic agent.
If a significant volume of the study drug infusion remains, restart the infusion at a more proximal site in the same limb or on the other side.

Formulation of HERCEPTIN®

HERCEPTIN® (lyophilized formulation) for use in this study will be supplied by the Sponsor, as a freeze-dried preparation. All HERCEPTIN® is supplied for parenteral IV administration; subcutaneous HERCEPTIN® is not permitted in this study. HERCEPTIN® is formulated in histidine, trehalose, and polysorbate 20. HERCEPTIN® for use in this study will be supplied by the Sponsor in vials containing a freeze-dried preparation for parenteral administration. For IV administration, each vial of HERCEPTIN® is reconstituted with Sterile Water for Injection (SWFI) dependent on the vial size, as follows:
HERCEPTIN® 440-mg vial is mixed with 20.0 mL of SWFI (not supplied)
HERCEPTIN® 150-mg vial is mixed with 7.2 mL of SWFI (not supplied)

Use of other reconstitution solvents is not allowed. The reconstituted solution contains 21 mg/mL trastuzumab and will be added to 250 mL of 0.9% sodium chloride injection for administration to the patient. None of the HERCEPTIN® formulations contains a preservative. The product is not intended to be stored after reconstitution and dilution unless this has taken place under aseptic conditions. Therefore, once the infusion is prepared, it is for single use only and should be administered promptly. The dose must be infused within 8 hours after reconstitution unless aseptically prepared and stored at 2° C.-8° C. (maximum refrigerated storage time is 24 hours). Each HERCEPTIN® vial provided for this study is to be used as a SINGLE DOSE VIAL ONLY. Each vial should not be used for more than one administration of Herceptin and not for more than 1 patient at a time. DO NOT FREEZE HERCEPTIN THAT HAS BEEN RECONSTITUTED.

Labeling of HERCEPTIN®

HERCEPTIN® will be labeled according to the regulatory requirements in each country, as well as in accordance with ICH Good Clinical Practice. The study Sponsor will provide Herceptin to all study sites labeled for investigational use only.

Storage of HERCEPTIN®

Vials of HERCEPTIN® are shipped with cool packs at a temperature ranging from 2° C. to 8° C. (36° F. to 46° F.) and must be placed in a refrigerator (same temperature range) immediately upon receipt to ensure optimal retention of physical and biochemical integrity. Temperature logs must be maintained (in accordance with local pharmacy practice) on the refrigerator to ensure proper storage conditions. Do not use beyond the use by date stamped on the vial. DO NOT FREEZE.

HERCEPTIN® may be sensitive to shear-induced stress (e.g., agitation or rapid expulsion from a syringe). DO NOT SHAKE. Vigorous handling of solutions of HERCEPTIN® results in aggregation of the protein and may create cloudy solutions. HERCEPTIN® should be carefully handled during reconstitution. Causing excessive foaming during reconstitution or shaking the reconstituted HERCEPTIN® may result in problems with the amount of HERCEPTIN® that can be withdrawn from the vial.

Preparation of HERCEPTIN®

Appropriate aseptic technique should be used when preparing the study drug. Each vial of HERCEPTIN® is reconstituted with SWFI as described above. HERCEPTIN® should be carefully handled during reconstitution. Causing excessive foaming during reconstitution or shaking the reconstituted HERCEPTIN® may result in problems with the amount of HERCEPTIN® that can be withdrawn from the vial.

The following instructions have to be followed:
1. Using a sterile syringe, slowly inject the sterile water for injection in the vial containing the lyophilized Herceptin, directing the stream into the lyophilized cake.
2. Swirl vial gently to aid reconstitution. DO NOT SHAKE!

Slight foaming of the product upon reconstitution is not unusual. Allow the vial to stand undisturbed for approximately 5 minutes. The reconstituted HERCEPTIN® results in a colorless to pale yellow transparent solution and should be essentially free of visible particulates.

Do not refrigerate or freeze HERCEPTIN® that has been reconstituted.

Drug Preparation: Dilution

The reconstituted solution will be added to an infusion bag containing 250 mL of 0.9% Sodium Chloride Injection, United States Pharmacopeia. Once the infusion is prepared, it should be administered immediately. If diluted aseptically, it may be stored for a maximum of 24 hours from reconstitution (do not store above 30° C.).

Dosage, Administration, and Compliance

Both Cohorts, A and B

In both cohorts, PERJETA® is given as a fixed non-weight-based dose of 840-mg IV loading dose, then 420 mg IV every 3 weeks. Herceptin is given as an 8-mg/kg IV loading dose, then 6 mg/kg IV every 3 weeks. The order of administration of PERJETA® and HERCEPTIN® is according to investigator preference. Chemotherapy should be given after PEREJTA® and HERCEPTIN®.

Treatment will continue as scheduled, or until investigator-assessed radiographic or clinical progression or recurrence of disease or unmanageable toxicity.

Weight should be recorded during screening and on Day 1 of each cycle for all patients. The baseline weight for a patient will be that measured on Cycle 1, Day 1. The amount of HERCEPTIN® to be administered must be recalculated if the patient's body weight has changed by >10% (increased or decreased) from the Cycle 1, Day 1 weight. The amount of HERCEPTIN® administered is calculated according to the patient's actual body weight, with no upper limit.

The amount of docetaxel, paclitaxel, doxorubicin, 5-fluorouracil, epirubicin, and cyclophosphamide is calculated according to the patient's BSA. The BSA and the amount of drug administered must be recalculated if the patient's body weight has changed by >10% (increased or decreased) from baseline. Recalculation of the amount of drug administered on the basis of smaller changes in body weight or BSA is at the investigators' discretion.

No dose reductions are allowed for PERJETA® or HERCEPTIN®. If the patient misses a dose of PERJETA® or HERCEPTIN® for any cycle and the time between doses is ≥6 weeks, a reloading dose of PERJETA® or HERCEPTIN® (840 mg and 8 mg/kg, respectively) should be given. Subsequent maintenance PERJETA® (420 mg) and HERCEPTIN® (6 mg/kg) doses will then be given every 3 weeks, starting 3 weeks later.

After surgery, patients continue to receive PERJETA® and HERCEPTIN® in the adjuvant setting until a total of 17 cycles of PERJETA® and HERCEPTIN® have been administered during the study. Adjuvant PERJETA® and HERCEPTIN® treatment should not start until 2 weeks after surgery. If the interval between the first dose of adjuvant Perjeta and Herceptin and the last dose of neoadjuvant PERJETA® and HERCEPTIN® exceeds 6 weeks, a reloading dose of 840 mg of PERJETA® and 8 mg/kg of HERCEPTIN® is required.

PERJETA®

The initial dose of PERJETA® will be administered over 60 (±10) minutes, and patients will be observed for a further 60 minutes. The infusion should be slowed or interrupted if the patient experiences infusion-related symptoms. If the infusion is well tolerated, subsequent doses may be administered over 30 (±10) minutes, and patients will be observed for a further 30 minutes for infusion-related symptoms such as fever or chills. All infusion-related symptoms must have resolved before HERCEPTIN® or chemotherapy is given or the patient is discharged. Patients who experience infusion-related symptoms may be premedicated with analgesics and antihistamines for subsequent infusions.

HERCEPTIN®

The initial dose of HERCEPTIN® will be administered over 90 (±10) minutes, and patients will be observed for at least 30 minutes from the end of the infusion for infusion-related symptoms such as fever or chills. Interruption or slowing of the infusion may help control such symptoms and may be resumed when symptoms abate. If the infusion is well tolerated, subsequent infusions may be administered over 30 (±10) minutes, and patients will be observed for a further 30 minutes. All infusion-related symptoms must have resolved before PERJETA® or chemotherapy is given or the patient is discharged. Patients who experience infusion-related symptoms may be premedicated with analgesics and antihistamines for subsequent infusions.

Cohort A (ddAC→T+PH)

Patients in Cohort A receive doxorubicin 60 mg/m² IV and cyclophosphamide 600 mg/m² IV every 2 weeks for four cycles (Cycles 1-4), followed 2 weeks later by weekly paclitaxel 80 mg/m² IV for 12 weeks (Cycles 5-8), with PERJETA® and HERCEPTIN® every 3 weeks from the start of paclitaxel (four cycles of PERJETA® and HERCEPTIN® in total during the neoadjuvant period). During ddAC, patients should receive G-CSF support according to local practice guidelines. After surgery, patients continue to receive PERJETA® and HERCEPTIN® in the adjuvant setting (Cycles 9-21) until a total of 17 cycles of PERJETA® and HERCEPTIN® have been given.

Doxorubicin

Doxorubicin will be administered at 60 mg/m² on Day 1 of each ddAC treatment. It may be given as an IV bolus over 3-5 minutes or as an infusion over 15-30 minutes. Dose delays and reduction for toxicity are permitted, and patients should receive G-CSF support according to local practice guidelines.

Cyclophosphamide

Cyclophosphamide will be administered at 600 mg/m² on Day 1 of each ddAC treatment. It should be given as an IV bolus over 3-5 minutes or as an infusion, in accordance with local policy. Patients with BSA of >2 m² should have their dose capped at 1200 mg. Dose delays and dose reductions for toxicity are permitted, and patients should receive G-CSF support according to local practice guidelines. Oral cyclophosphamide is not permitted.

Paclitaxel

Paclitaxel will be administered at a dose of 80 mg/m² as an IV infusion over a minimum of 1 hour. When given on the same day, it should be given after PERJETA® and HERCEPTIN®. Premedication including corticosteroids should be administered as clinically indicated according to routine practice.

Cohort B (FEC→D+PH)

Patients in Cohort B receive 5-fluorouracil 500 mg/m² IV, epirubicin 100 mg/m² IV, and cyclophosphamide 600 mg/m² IV, every 3 weeks for four cycles (Cycles 1-4), followed (3 weeks later) by docetaxel every 3 weeks (75 mg/m² for the first dose, and 100 mg/m² for subsequent doses if no dose-limiting toxicity occurs) for four cycles (Cycles 5-8), with PERJETA® and HERCEPTIN® every 3 weeks from the start of docetaxel (Cycles 5-8; i.e., four cycles of PERJETA® and HERCEPTIN® in total during the neoadjuvant period). After surgery, patients continue to receive PERJETA® and HERCEPTIN® in the adjuvant setting (Cycles 9-21) until a total of 17 cycles of PERJETA® and HERCEPTIN® have been given.

5-Fluorouracil

5-fluorouracil 500 mg/m² will be administered on Day 1 of each cycle of FEC treatment as an IV bolus or infusion, in accordance with local policy. Patients with BSA of >2 m² should have their dose capped at 1200 mg. Dose delays and dose reductions for toxicity are permitted.

Epirubicin

Epirubicin 100 mg/m² will be administered on Day 1 of each cycle of FEC treatment as an IV bolus over 3-5 minutes or as an infusion over 1-30 minutes, in accordance with local policy. Dose delays and dose reductions for toxicity are permitted.

Cyclophosphamide

Cyclophosphamide 600 mg/m² will be administered on Day 1 of each cycle of FEC treatment as an IV bolus over 3-5 minutes or as an infusion, in accordance with local policy. Patients with BSA of >2 m² should have their dose capped at 1200 mg. Dose delays and dose reductions for toxicity are permitted. Oral cyclophosphamide is not permitted.

Docetaxel

Docetaxel is administered as an IV infusion over 60 (±10) minutes, after PERJETA® and HERCEPTIN®, at a starting dose of 75 mg/m² for the first cycle (Cycle 5). At the investigator's discretion, the dose may be escalated to 100 mg/m² for subsequent cycles (Cycles 6 to 8) provided no dose-limiting toxicity occurs.

Premedication, including corticosteroids, should be administered according to routine practice. Patients must be closely observed from the start of the infusion for hypersensitivity reactions which may occur within minutes. Severe hypotension, bronchospasm, or generalized rash/erythema requires immediate discontinuation of docetaxel and appropriate treatment. The infusion may be slowed for minor symptoms, such as flushing or local cutaneous reactions. Patients experiencing severe hypersensitivity reactions should be discontinued from study treatment but maintained in the schedule of assessments unless consent is withdrawn. Premedication consisting of a corticosteroid may be given according to institutional guidelines. Similarly, prophylactic G-CSF may be used to mitigate the risk of hematologic toxicities according to local policies. Treatment of neutropenia with G-CSF is permitted according to local policies. In all cases, G-CSF will not be considered as a study drug and will not be provided by the Sponsor.

Concomitant Therapy

Surgery

Patients in both cohorts are scheduled to undergo surgery after eight cycles of neoadjuvant therapy. For patients in Cohort A, the eight cycles will take about 20 weeks, and for patients in Cohort B, about 24 weeks. Patients may undergo BCS or mastectomy according to routine clinical practice. The reasons for choosing BCS or mastectomy will be recorded prior to surgery and a copy of the pathology report will be provided.

Before starting neoadjuvant treatment, the primary tumor site should be marked using the method which is standard locally (for example, skin tattoo or surgical clip) to enable appropriate surgical excision in case of tumor regression during neoadjuvant therapy.

The following guidelines on sentinel lymph node biopsy (SLNB) are based on 2013 NCCN and ESMO guidelines (Senkus et al. 2013; Theriault et al. Journal of the National Comprehensive Cancer Network, 2013, Vol. 11, No. 7, 753-761). However, ongoing clinical trials are evaluating the role of axillary radiotherapy as an alternative to axillary dissection, and identifying subgroups of patients who may be able to omit axillary dissection following a positive SLNB. As a result, these broad guidelines may be superseded. Investigators may follow more up-to-date guidelines based on emerging data once they have been incorporated into institutional, local, national, or international guidelines (e.g., NCCN, ESMO, St Gallen, Lisbon Conference, or American Society of Clinical Oncology Clinical Practice Guidelines). Some recommendations follow below; however, investigators may follow local practice guidelines.

Where possible, patients with clinically node-positive disease prior to neoadjuvant therapy should undergo an ultrasound-guided fine needle aspiration or core needle biopsy to confirm nodal involvement prior to commencement of neoadjuvant therapy. Patients with confirmed node-positive disease should undergo a Level I and II axillary dissection at the time of definitive surgery (after neoadjuvant therapy). It is recommended that at least 10 lymph nodes be removed for pathologic examination.

SLNB is the preferred method of axillary lymph node staging for patients with clinically node-negative disease, if an experienced team is available. SLNB may be conducted before or after neoadjuvant therapy according to routine practice. If SLNB is not available, the patient should undergo a Level I and II axillary dissection at the time of definitive surgery (after neoadjuvant therapy). It is recommended that at least 10 lymph nodes be removed for pathologic examination.

If SLNB is available, the following guidelines apply.
For patients with clinically node-negative disease prior to neoadjuvant therapy who undergo SLNB prior to neoadjuvant therapy:

If the sentinel lymph node (SLN) is histologically negative, axillary dissection may be omitted at the time of definitive surgery (after neoadjuvant therapy).

If the SLN is histologically positive, the patient should undergo a Level I and II axillary dissection at the time of definitive surgery (after neoadjuvant therapy).
For patients with clinically node-negative disease prior to neoadjuvant therapy who do not undergo SLNB prior to neoadjuvant therapy:

An SLNB may be performed at the time of definitive surgery (after neoadjuvant therapy). If the SLN is histologically positive, then a Level I and II axillary dissection should be performed.

For sentinel nodes involving the internal mammary chain, refer to local, national, or international guidelines.

Level III axillary dissections should only be performed for patients with gross disease in the Level II nodes.

Radiotherapy

Before actively enrolling patients, each center must define a radiotherapy policy for treating patients in the trial. Radiotherapy is given after chemotherapy and surgery, during adjuvant antibody therapy (and hormone therapy, if indicated).

Hormone Therapy

Before actively enrolling patients, each center must set a policy for the use of tamoxifen, ovarian ablation, or both for patients in the trial. Study sites must also set their local policy for the use of registered aromatase inhibitors. Aromatase inhibitors will be allowed as adjuvant hormone therapy for postmenopausal patients who are hormone receptor-positive, in countries where it has been registered for this indication. Its use must be consistent with the registered label. Hormone therapy is given after chemotherapy and surgery, during adjuvant antibody therapy.

No other hormone therapy for primary breast cancer is allowed, including pure anti-estrogens and progestational agents, unless it becomes approved for adjuvant therapy during the conduct of the trial.

### Study Assessments

### Description of Study Assessments

Core Biopsy

The diagnosis of primary breast cancer will be performed as per local standard of care.

Submission of tumor tissue from the core biopsy of the primary tumor (preferred) or involved lymph node (if primary tumor cannot be biopsied) is mandatory for the trial. The tissue will be used to confirm HER2 and estrogen receptor (ER)/progesterone receptor (PgR) status and for subsequent biomarker research (i.e., molecular subtyping). Samples must be formalin-fixed and paraffin-embedded, and tumor blocks are preferred. If it is not possible to submit tumor blocks, sites must provide 15 freshly cut slides.

A 14-gauge needle is recommended, using an automatic device fired 3-4 times into the lesion to collect sufficient tumor tissue.

Submission of tissue obtained at surgery from resection specimens from patients with residual disease is encouraged. These samples must be submitted as FFPE tissue blocks; slides cannot be accepted.

HER2 Screening for Eligibility and Central Assessment of Hormone Receptor Status

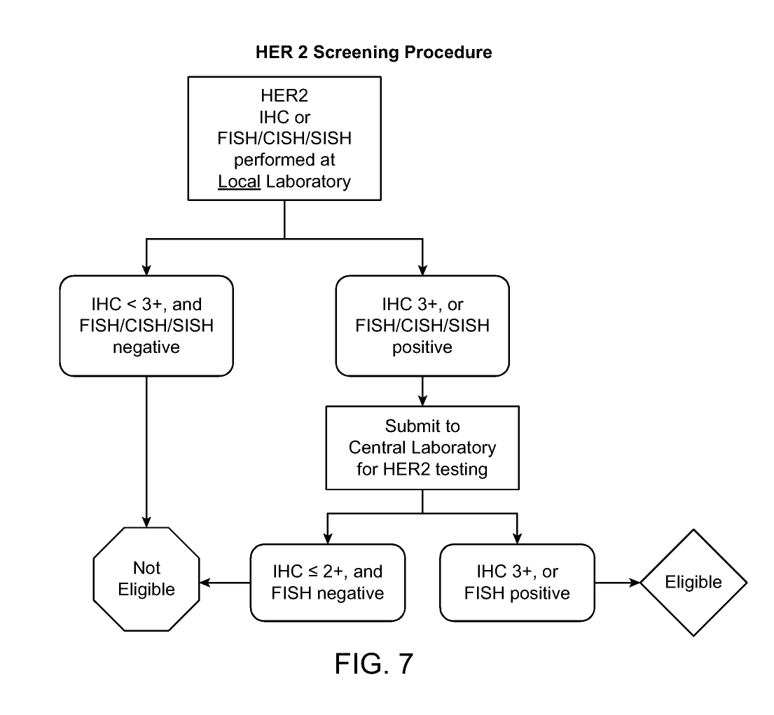

Patients should be initially screened for HER2 status by the local laboratory and should have an HER2 score of 3+ by immunohistochemistry (IHC) or HER2 (c-erbB2) gene amplification by in situ hybridization (FISH, SISH, or CISH) to qualify for central laboratory screening (see FIG. 7).

For central confirmation, HER2 positivity is defined as IHC 3+ in >10% of immunoreactive cells or c-erbB2 gene amplification by ISH (ratio of c-erbB2 gene signals to centromere 17 signals ≥2.0).

Central laboratory confirmation of a positive HER2 status is required prior to enrollment in the study. The outcome of this assessment will be communicated to the investigator.

In addition, central assessment of hormone receptor status (ER and PgR) will be conducted according to American Society of Clinical Oncology/College of American Pathologists guidelines (Hammond et al. 2010). The results will be communicated to the investigator. The investigator may treat the patient with adjuvant hormone therapy according to local or central results, but central results of hormone receptor status will be used in data analyses for the study.

Only patients who are HER2-positive by central determination will be allowed to enter the study; patients with overall negative and equivocal scores will be excluded from entry into the study.

Cardiac Function

All patients must have an LVEF measurement of at least 55% by ECHO (preferably) or MUGA scan prior to enrollment. Investigators must be aware of local institutional regulations regarding the maximum allowable frequency of repeat MUGA scans. The repeated administration of radioisotopes is limited in some nuclear medicine laboratories, and patients in this study require monitoring on more than four occasions within 1 year.

Patients must also have an assessment for history of cardiac events, a physical examination, and a baseline electrocardiogram (ECG) prior to enrollment to exclude any cardiac condition that would render them ineligible for participation in this trial. An ECG will also be performed after completion of anthracycline chemotherapy (and prior to the first cycle of PERJETA®/HERCEPTIN®/taxane) and thereafter as clinically indicated.

Laboratory Assessments

Samples for the following standard laboratory tests will be sent to the study site's local laboratory for analysis:
Hematology: complete blood count, WBC count, RBC count, hemoglobin, hematocrit, platelet count, differential count (neutrophils, eosinophils, basophils, monocytes, lymphocytes, other cells). During adjuvant PERJETA® and HERCEPTIN® treatment, complete blood counts, including differential and platelets, are scheduled as per local medical practice for adjuvant Herceptin monotherapy.
Serum chemistry: sodium, potassium, chloride, bicarbonate, glucose, BUN or urea, creatinine, total protein, albumin, phosphorus, calcium, total and direct/indirect bilirubin (if needed), alkaline phosphatase, ALT, AST, uric acid, LDH. Limited serum chemistry includes only alkaline phosphatase, AST, ALT, LDH, total and direct/indirect bilirubin, and creatinine Bilirubin fractions (direct and indirect) need to be measured only if total bilirubin is greater than ULN.
Coagulation (INR, aPTT, PT)
Pregnancy test: All women of childbearing potential (premenopausal women and for women less than 12 months after the onset of menopause, unless they have undergone surgical sterilization) will have a serum pregnancy test at a local laboratory within 7 days prior to the first administration of study medication. Women who have undergone surgical sterilization or are postmenopausal are exempt from pregnancy assessments.
Urinalysis by dipstick (pH, specific gravity, glucose, protein, ketones, blood) with microscopic examination (for sediment, RBCs, WBCs, casts, crystals, epithelial cells, bacteria) if clinically indicated.

Clinical Tumor Response Evaluations

Clinical breast examination (CBE) includes examination of the breast, axilla, and supraclavicular fossa. Patients with breast tumors ≥2 cm at baseline will have their clinical response assessed as CR, PR, SD, or PD, as determined by mammogram and CBE, and/or other methods of evaluation. Patients whose disease does not meet this criterion (i.e., those with only node positive disease and tumors <2 cm) will have their clinical response assessed as CR, SD, or PD (but not as PR, since the tumors are too small to measure this response accurately), as determined by mammogram and/or CBE, and/or other methods of evaluation. All patients, irrespective of the size and measurability of the primary tumor and locoregional lymph nodes, are evaluable for disease progression by CBE and/or mammography, and/or other methods of evaluation and will be included in the calculation of clinical response rate.

During the neoadjuvant treatment period (prior to surgery), tumor response will be assessed using CBE and/or mammography and/or other methods of evaluation as per local medical practice according to the Schedule of Assessments. When evaluating response by CBE, lesions should be measured in two dimensions, and response assessed according to the principles of RECIST v1.1 criteria (Eisengauer et al. 2009). Provided that the patient's clinical status has not changed, the screening mammogram can be performed up to 42 days prior to the start of treatment. The mammogram at screening, presurgery and final visit/withdrawal can be replaced by MRI at the investigator's discretion, but the same method of assessment must be used throughout for an individual patient.

After completion of neoadjuvant therapy and prior to surgery, a further tumor response assessment is required, including a CBE and mammogram, and/or other methods of evaluation.

After surgery, CBE will continue according to the Schedule of Assessments to detect signs of locoregional relapse.

Results of any additional methods of assessment (such as ultrasound, CT, X-rays, or MRI) may be included in the assessment of response as per routine practice (results of these modalities will be collected in the eCRF). Consistency of consecutive mammograms, CBE, CT scans, X-rays, or MRIs should be ensured during all assessments for each patient, with the same technique being used for evaluating the target lesion throughout the treatment period, whenever possible. Tumor measurements should be made by the same investigator or radiologist for each patient during the study to the extent that this is feasible. In case of clinically measurable superficial (such as skin) lesions, repeated photographs should be used to document tumor response. These photos must include a ruler for documentation purposes.

If the lesion shows clear signs of progression, the patient will be withdrawn from study treatment and provided with the local standard of care.

Discovery of ipsilateral or contralateral DCIS or LCIS during neoadjuvant treatment period will not be considered progressive disease. However, invasive contralateral breast carcinoma will be classified as progressive disease.

Clinical responses will be assessed locally and will not be independently reviewed.

Pathologic Response Evaluation

Pathologic response will be assessed by the local pathologist using guidelines provided in a Pathology Manual. A complete pathologic response is defined as the absence of invasive disease in the breast and axilla (tpCR; i.e., ypT0 or ypTis, ypN0) based on microscopic examination of the surgical specimen following neoadjuvant therapy. Complete pathologic response rate is the main efficacy endpoint of the study.

Copies of the Pathology Report(s) from the patient's primary (main) surgery must be submitted to the Sponsor within 6 weeks of the date of surgery. If additional information on lymph nodes at surgery is present in other reports, these should also be submitted to the Sponsor.

Diagnosis of Relapse or Recurrence

Recurrent disease includes: local, regional, and distant recurrence and contralateral breast cancer. Patients who are diagnosed with in situ breast disease or second (non-breast) malignancies should be maintained in regular follow-up wherever possible to fully capture any subsequent recurrent disease events. In cases of diagnostic doubt (e.g., ill-defined, palpable mass in an irradiated breast), histologic or cytologic confirmation of recurrence should be obtained whenever possible.

Some patients may develop a suspicious recurrence that leads quickly to death without the possibility of confirming relapse of disease. Efforts should be made to obtain an autopsy report in such patients.

The earliest date of diagnosis of recurrent disease should be used and recorded. This should be based on clinical, radiological, histological, or cytological evidence. The date of disease recurrence should be reported as the date of first diagnosis of a lesion (i.e., an objective finding), not the date of occurrence of the first symptom.

All second primary malignancies are to be reported whenever they occur during the study. Patients diagnosed with a second primary malignancy not requiring systemic therapy (i.e., chemotherapy, hormonal therapy, targeted therapy, etc.) and with no evidence of breast cancer recurrence will remain on study and should continue with study drug treatment according to the protocol and schedule of assessments, if considered by the investigator to be in the patient's best interest, whenever possible.

The following events are NOT considered recurrent disease, but must be recorded.
Ipsilateral and contralateral LCIS
Ipsilateral and contralateral DCIS
Carcinoma in situ of the cervix
Basal or squamous cell carcinoma of the skin

Following recurrence, all patients should be followed for survival according to the Schedule of Assessments. In addition, LVEF assessments should continue to be performed every 6 months for 2 years, and then annually for an additional 2 years. Heart failure occurring at any time during the study and up to 5 years after the last patient was enrolled must be reported irrespective of the initiation of alternative treatment and irrespective of any causal relationship. Pregnancy tests should also continue and pregnancies should be reported until 7 months after the last dose of study treatment, irrespective of disease progression or relapse or the initiation of alternative treatment. Related serious adverse events and non-breast second primary malignancies (reportable as serious adverse events) should also be reported until the end of the study.

Mandatory Serum Samples for Anti-Therapeutic Antibody, Pertuzumab, and Biomarker Analysis

Blood samples (10 mL) will be collected at baseline and subsequent time points and divided into three aliquots of serum: One aliquot to measure serum pertuzumab ATAs, one aliquot to measure serum pertuzumab concentrations, and one aliquot for biomarker research. These are mandatory samples for the trial.

ATAs to pertuzumab in serum will be detected using a validated bridging ELISA method that is based on the formation of bridging antibody complexes with labeled pertuzumab molecules. This assay utilizes pertuzumab labeled with biotin and pertuzumab labeled with digoxigenin. A validated ELISA will used to measure pertuzumab concentrations to further characterize the ATA results.

The aliquot reserved for biomarker research may be used for the identification of dynamic (non-inherited) biomarkers. This research may help to better understand the association between biomarkers and efficacy, adverse events, or disease progression, to better understand the disease biology, or to improve diagnostic tests. Analysis of these samples may include, but not be limited to, circulating tumor DNA, circulating proteins or peptides (such as ligands of HER family of receptors) or potential markers of cardiac damage.

Samples will be destroyed no later than 5 years after the date of final closure of the associated clinical database.

Management of Specific Adverse Events

No evidence available at the time of finalization of this study protocol indicated that special warnings and precautions were appropriate, other than those noted in the PERJETA® IB and prescribing information.

PERJETA® should only be initiated under supervision of a physician experienced in the treatment of cancer patients.

Allergic Reactions, Including Anaphylaxis and Infusion Associated Symptoms

Like other monoclonal antibodies, PERJETA® has been associated with infusion-associated symptoms (such as chills, diarrhea, fatigue, headache, nausea, and pyrexia) and hypersensitivity reactions. The administration of PERJETA® should be performed in a setting with emergency equipment and staff who are trained to monitor medical situations and respond to medical emergencies. Patients will be monitored during each PERJETA® infusion and at least 60 minutes following the completion of the first infusion for any adverse effects. If infusion-associated symptoms occur, patients should be monitored until complete resolution of signs and symptoms. Patients who experience infusion-associated symptoms may be managed by slowing or interrupting the infusion and by providing supportive care with oxygen and medications (e.g., beta-agonists, antihistamines, antipyretics, or corticosteroids), as determined by the investigator to be clinically appropriate. Patients who experience infusion-associated symptoms may subsequently be premedicated with analgesia and antihistamines. If the infusion is well tolerated, patients will be observed for 30 minutes following subsequent infusions.

The infusion of PERJETA® should be stopped in patients who develop dyspnea or clinically significant hypotension (defined as per investigator discretion). Patients who experience an NCI CTCAE Grade 3 or 4 allergic reaction or acute respiratory distress syndrome (ARDS) should be discontinued from treatment.

Cardiotoxicity

LVEF declines have been reported with drugs that block HER2 activity, including PERJETA® and HERCEPTIN®.

To enter this study, all patients must have a LVEF (by ECHO or MUGA scan) of ≥55%.

Patients in both Cohorts A and B should not start anti-HER2 drugs if his or her LVEF is <50% after anthracyclines therapy.

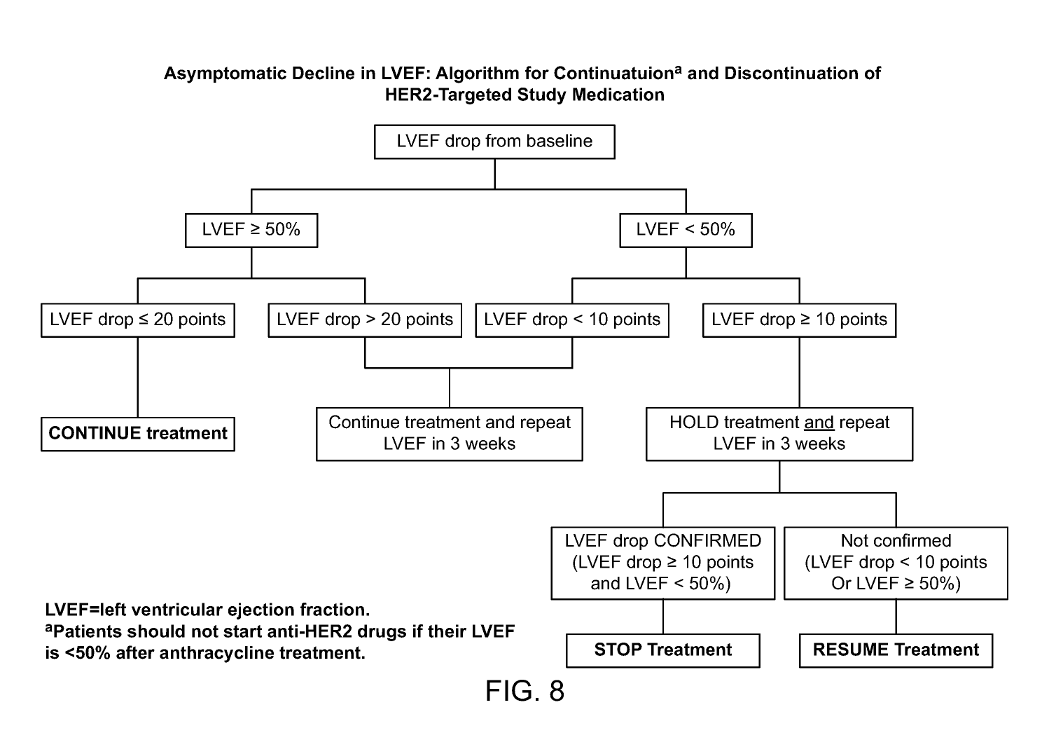

Patients who experience an asymptomatic decrease in LVEF after anthracycline therapy may continue to receive the taxane component of chemotherapy at the discretion of the investigator. HER2-targeted therapy may be subsequently initiated (or restarted) in accordance with the algorithm in FIG. 8; the delay in initiating (or restarting) HER2-targeted therapy should not exceed 6 weeks. Regular monitoring of LVEF is required thereafter, according to set Schedule of Assessments.

If severe symptomatic heart failure develops (NYHA Class III or IV) or there is a significant LVEF decrease (LVEF decline ≥10 percentage points to an LVEF value <50%), the patient must discontinue anti-HER2 therapy. Heart failure or left ventricular dysfunction should be treated and monitored according to standard medical practice. These patients should be evaluated by a certified cardiologist and the results of this evaluation should be reported on the eCRF.

FIG. 8 summarizes the management of study medication in patients who develop an asymptomatic decrease in LVEF. The decision to initiate HER2-targeted therapy (for patients in Cohort B) and whether to continue or stop therapy (for patients in both cohorts) should be based on two factors: measured LVEF and changes in LVEF from baseline.

Patients who discontinue PERJETA® and HERCEPTIN® for heart failure or LVEF decline should continue to undergo LVEF assessments irrespective of the initiation of alternative systemic anti-cancer therapy until resolution, improvement to baseline status, no further improvement can be expected, or death. Additional LVEF assessments may be required for these patients (beyond those specified in the Schedule of Assessments), according to the investigator's clinical judgment. The results of these assessments should be reported.

Adverse Events

According to the ICH guideline for Good Clinical Practice, an adverse event is any untoward medical occurrence in a clinical investigation subject administered a pharmaceutical product, regardless of causal attribution. An adverse event can therefore be any of the following:
Any unfavorable and unintended sign (including an abnormal laboratory finding), symptom, or disease temporally associated with the use of a medicinal product, whether or not considered related to the medicinal product
Any new disease or exacerbation of an existing disease (a worsening in the character, frequency, or severity of a known condition), except events that are clearly consistent with the expected pattern of recurrence or progression of the underlying disease should not be recorded as adverse events.
Recurrence of an intermittent medical condition (e.g., headache) not present at baseline
Any deterioration in a laboratory value or other clinical test (e.g., ECG, X-ray) that is associated with symptoms or leads to a change in study treatment or concomitant treatment, or discontinuation from study drug
Adverse events that are related to a protocol-mandated intervention, including those that occur prior to assignment of study treatment (e.g., screening invasive procedures, such as biopsies)

Serious Adverse Events (Immediately Reportable to the Sponsor)

A serious adverse event is any adverse event that meets any of the following criteria:
Fatal (i.e., the adverse event actually causes or leads to death)
Life threatening (i.e., the adverse event, in the view of the investigator, places the patient at immediate risk of death)

This does not include any adverse event that might have caused death if it had occurred in a more severe form or was allowed to continue.
Requires or prolongs inpatient hospitalization
Results in persistent or significant disability or incapacity (i.e., the adverse event results in substantial disruption of the patient's ability to conduct normal life functions)
Congenital anomaly or birth defect in a neonate or infant born to a mother exposed to study drug
Significant medical event in the investigator's judgment (e.g., may jeopardize the patient or may require medical or surgical intervention to prevent one of the outcomes listed above)

The terms “severe” and “serious” are not synonymous. Severity refers to the intensity of an adverse event (e.g., rated as mild, moderate, or severe, or according to NCI CTCAE criteria; see the following Table 1); the event itself may be of relatively minor medical significance (such as severe headache without any further findings).

Severity and seriousness need to be independently assessed for each adverse event recorded on the eCRF.

Serious adverse events are required to be reported by the investigator to the Sponsor immediately (i.e., no more than 24 hours after learning of the event).

TABLE 1
Adverse Event Severity Grading Scale

| Grade | Severity |
|---|---|
| 1 | Mild; asymptomatic or mild symptoms, clinical or diagnostic |
| | observations only, or intervention not indicated. |
| 2 | Moderate; minimal, local, or non-invasive intervention |
| | indicated; or limiting age-appropriate instrumental activities |
| | of daily living.^{a} |
| 3 | Severe or medically significant, but not immediately life- |
| | threatening; hospitalization or prolongation of hospitalization |
| | indicated, disabling, or limiting self-care activities |
| | of daily living^{b,c} |
| 4 | Life-threatening consequences or urgent intervention indicated^{d} |
| 5 | Death related to adverse event^{d} |

NCI CTCAE = National Cancer Institute Common Terminology Criteria for Adverse Events.
Note:
Chart based on the most recent version of NCI CTCAE (version 4.0), which can be found at: http://ctep.cancer.gov/protocolDevelopment/electronic_applications/ctc.htm
^{a}Instrumental activities of daily living refer to preparing meals, shopping for groceries or clothes, using the telephone, managing money, etc.
^{b}Examples of self-care activities of daily living include bathing, dressing and undressing, feeding one's self, using the toilet, and taking medications, as performed by patients who are not bedridden.
^{c}If an event is assessed as a significant medical event, it must be reported as a serious adverse event,
^{d}Grade 4 and 5 events must be reported as serious adverse events).

Non-Serious Adverse Events of Special Interest (Immediately Reportable to the Sponsor)

Non-serious adverse events of special interest are required to be reported by the investigator to the Sponsor immediately (i.e., no more than 24 hours after learning of the event). Adverse events of special interest for this study include the following:
Cases of potential drug-induced liver injury that include an elevated ALT or AST in combination with either an elevated bilirubin or clinical jaundice, as defined by Hy's law
Suspected transmission of an infectious agent by the study drug, defined as:

Any organism, virus, or infectious particle (e.g., prion protein-transmitting transmissible spongiform encephalopathy), pathogenic or non-pathogenic, is considered an infectious agent. A transmission of an infectious agent may be suspected from clinical symptoms or laboratory findings indicating an infection in a patient exposed to a medicinal product. This term only applies when a contamination of the study drug is suspected.
An asymptomatic decline in LVEF requiring treatment or leading to discontinuation of PERJETA® and HERCEPTIN®.

Selected Adverse Events

Heart Failure

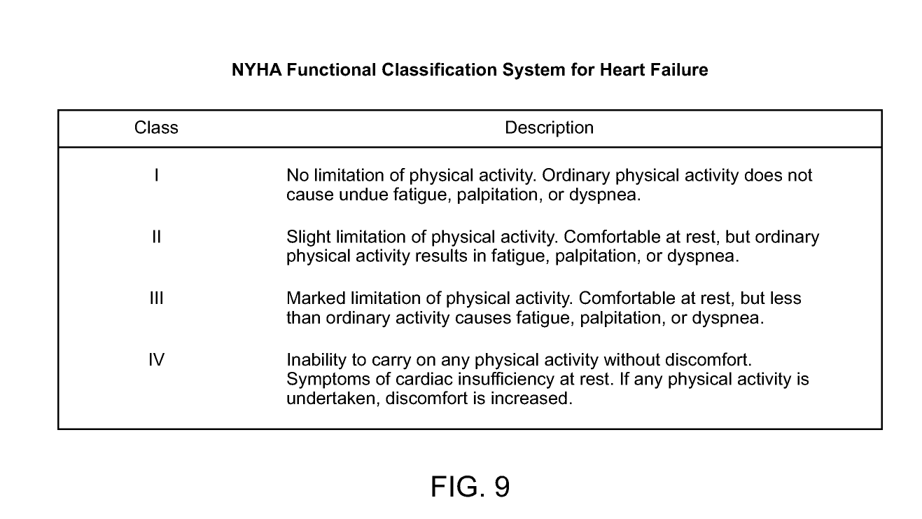

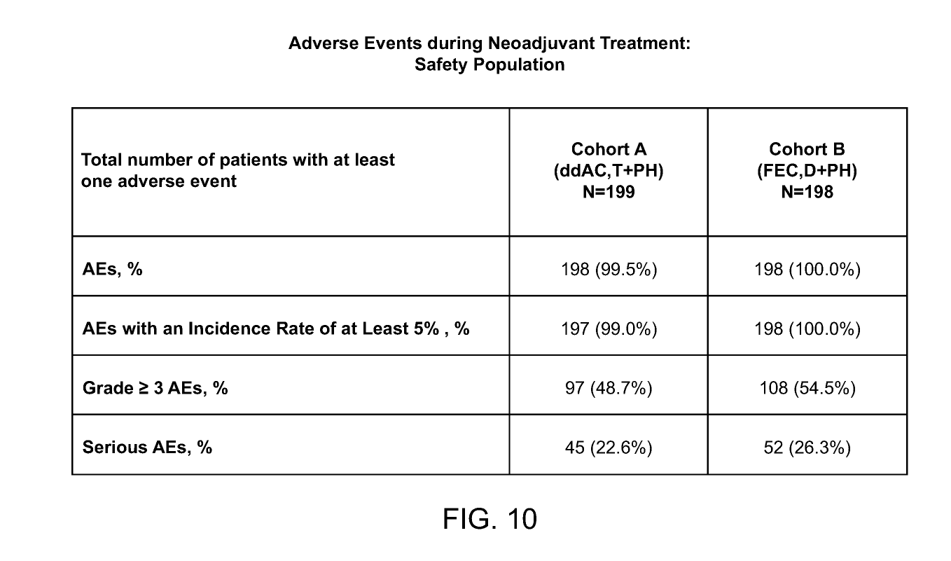

Symptomatic left ventricular systolic dysfunction (otherwise referred to as heart failure) should be reported as a serious adverse event. If the diagnosis is heart failure, it should be reported as such, and not as individual signs and symptoms of heart failure. In the eCRF, signs and symptoms should be recorded. A cardiac consultation is recommended for patients who develop symptomatic left ventricular systolic dysfunction (heart failure). Heart failure should be graded according to NCI CTCAE v4.0 (Grade 2, 3, 4, or 5), as well as according to the NYHA classification (Class II, III, and IV; see FIG. 9). Left ventricular systolic dysfunction should not be used to describe symptomatic dysfunction, as per NCI CTCAE v4.0.

Heart failure occurring during the study and up to 5 years after the last patient enrolled must be reported irrespective of causal relationship and followed until one of the following occurs: resolution or improvement to baseline status, no further improvement can be expected, or death.

Asymptomatic Declines in Left Ventricular Ejection Fraction

Asymptomatic declines in LVEF should not be reported as adverse events because LVEF data are collected separately in the eCRF. Exceptions to this rule are as follows:
An asymptomatic decline in LVEF of ≥10 percentage-points from baseline to an LVEF <50% must be reported as an adverse event with the term of ejection fraction decreased, as per NCI CTCAE v4.0. In addition, a comment in the adverse events comments field should confirm that the event was asymptomatic.

Safety Analyses

The safety objectives will be assessed in the safety population.

The primary objective of this study is to evaluate the cardiac safety of neoadjuvant treatment with each of the two treatment regimens. Cardiac safety will be evaluated by assessment of the following endpoints:
The incidence of NYHA Class III and IV heart failure and the associated 95% CIs will be calculated for each treatment cohort during the neoadjuvant period (primary objective), and adjuvant and follow-up periods.
The incidence of LVEF declines (of ≥10%-points from baseline and to a value of <50%) with the associated 95% CIs will be calculated for each treatment cohort during the neoadjuvant period (primary objective), and adjuvant and follow-up periods.

The Clopper-Pearson method for binomial proportions will be used to derive the 95% CIs.

The secondary safety objectives are to evaluate the safety profiles of the two treatment regimens during the neoadjuvant, adjuvant, and follow-up periods, and will be assessed as follows:
The incidence and severity of adverse events and serious adverse events will be summarized and reported.
Laboratory test abnormalities will be summarized and reported.
Serum levels and the incidence of ATAs to pertuzumab and their relationship to safety events and efficacy will be summarized and reported.

Safety will also be summarized in selected subgroups for selected time periods (e.g., cardiac safety in Cohort B before commencement of PERJETA® and HERCEPTIN®).

Main Efficacy Endpoint

Cardiac safety is the primary objective of the study, so all efficacy analyses are considered secondary or exploratory.

The main efficacy endpoint is the rate of pCR in the breast and nodes (ypT0/is ypN0 tpCR) evaluated after surgery following a scheduled eight cycles of neoadjuvant treatment, lasting approximately 21 weeks in Cohort A (ddAC→T+PH) and approximately 25 weeks in Cohort B (FEC→D+PH). Patients who do not undergo surgery or do not have a valid tpCR assessment will be considered non-responders in the analysis.

For each treatment cohort, the observed rate and Clopper-Pearson 95% CI will be calculated.

Secondary Efficacy Endpoints

Clinical response rate prior to surgery will be summarized and reported. For patients who have clinical response assessed during neoadjuvant therapy but not immediately prior to surgery, and patients who do not undergo surgery, the last recorded clinical response assessment will be considered in the analysis. Patients without any assessment of clinical response prior to surgery will be considered non-responders in the analysis.

EFS is defined as the time from enrollment to the first occurrence of progressive disease, relapse, or death from any cause. Ipsilateral or contralateral in situ disease and second primary non-breast cancers (including in situ carcinomas and non-melanoma skin cancers) will not be counted as progressive disease or relapse. Patients who are withdrawn from the study without documented progression or relapse and for whom there exists eCRF evidence that evaluations have been made, will be censored at the date of the last assessment at which the patient was known to be free from progressive disease or relapse. Patients with no tumor evaluations after baseline will be censored at the date of enrollment plus 1 day.

iDFS is defined as the time from the first date of no disease (i.e., the date of surgery) to the first documentation of progressive invasive disease, relapse, or death. Ipsilateral or contralateral in situ disease and second primary non-breast cancers (including in situ carcinomas and non-melanoma skin cancers) will not be counted as progressive disease or relapse. Patients who are withdrawn from the study without documented progression or relapse and for whom there exists eCRF evidence that evaluations have been made, will be censored at the date of the last assessment at which the patient was known to be alive and disease-free. Patients with no postbaseline information and patients who do not undergo surgery will be excluded from the analysis. It should be noted that this definition of iDFS (which excludes second primary non-breast cancers as events) is the same as that used in the APHINITY trial and is not the same as iDFS defined by Hudis et al. (2007) in which second primary non-breast cancers are counted as events.

OS is defined as the time from enrollment to death from any cause. Patients who are alive or lost to follow-up will be censored at their last known date in the study. Patients with no post-baseline assessments will be censored at the date of enrollment plus 1 day.

The Kaplan-Meier approach will be used to plot EFS, iDFS, and OS, as well as to estimate the proportion of patients who are event-free at landmark time points for each treatment cohort.

Exploratory Analyses

Exploratory analyses will include the analysis of tpCR rates with respect to baseline factors (e.g., hormone receptor status) and the calculation of bpCR and GBG pCR rates.

The BCS rate and 95% CI will be summarized for each treatment cohort for the following three patient populations:
All female patients in the ITT population
All female patients with T2 or T3 tumors at study entry
All female patients with T2 or T3 tumors at study entry for whom mastectomy was planned

The percentage of patients who are potentially eligible for BCS and who did not undergo BCS will be summarized, as well as the reasons for this decision (at baseline and at the time of surgery).

The re-excision surgery rate will be summarized for each treatment cohort, and will be calculated in the subset of the ITT population who receive BCS.

All biomarker analyses will be exploratory or descriptive in nature. pCR will be summarized by molecular-defined breast cancer subtypes. In the absence of a PERJETA®-naive control arm, the predictive value of biomarkers cannot be derived with respect to PERJETA® treatment benefit.

Results

The Primary Objectives of the study were to assess:
Incidence of NYHA Class III and IV heart failure during the neoadjuvant period, as assessed by the investigator
Incidence of clinically significant LVEF declines (≥10%-points from baseline and to a value of <50%) during the neoadjuvant period.

Secondary Objectives:
tpCR, defined as eradication of invasive disease in the breast and axilla (i.e. ypT0/is ypN0), and according to the local pathologist's assessment.
Incidence and severity of other adverse events and serious adverse events
Laboratory test abnormalities
Incidence of anti-therapeutic antibodies (ATA) to pertuzumab and their relationship to safety events.

Exploratory Objectives:
To assess the pCR rate according to alternative definitions (bpCR, German Breast Group [GBG] pCR, and Residual Cancer Burden [RCB] index)
To assess pCR rates according to subtypes of breast cancer defined by molecular profiles; e.g., the intrinsic subtypes of breast cancer defined by the PAM50 classifier
To document the rate of BCS for female patients in the study and for female patients with T2 or T3 tumors
To document the rate of re-excision surgery for residual disease

FIG. 10 presents a summary of Adverse Events (AEs) during neoadjuvant treatment in the safety population for Cohorts A and B. AEs with an incidence rate of at least 5%, Grade ≥3 AEs and Serious AEs are separately shown.

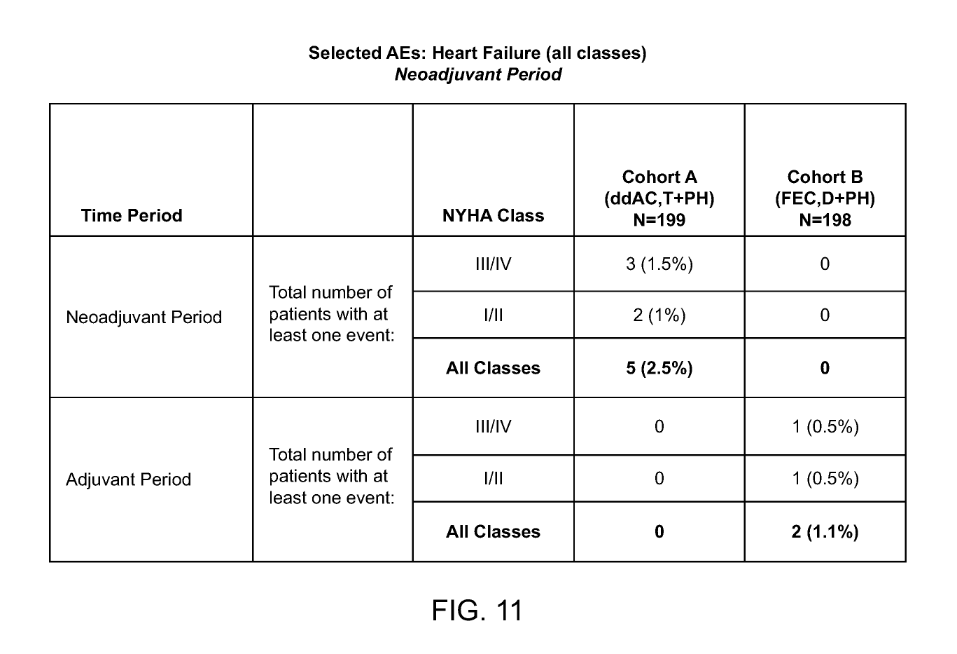

FIG. 11 presents select AEs: Heart Failure (all classes), during the neoadjuvant period and adjuvant period.

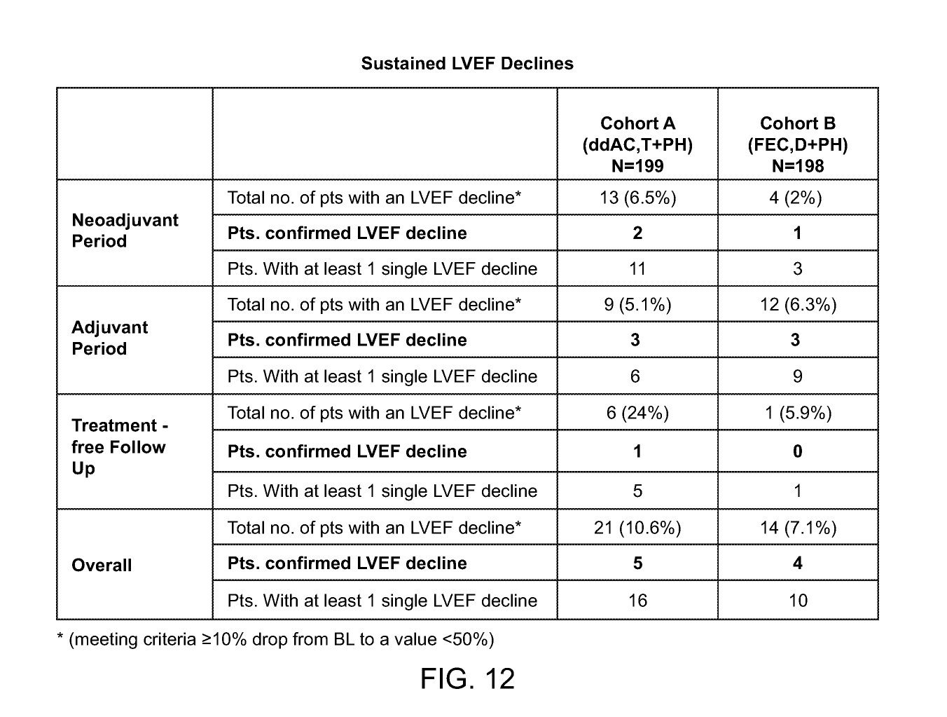

FIG. 12 is a summary table of sustained LVEF Declines during the neoadjuvant period, adjuvant period, and treatment-free follow up.

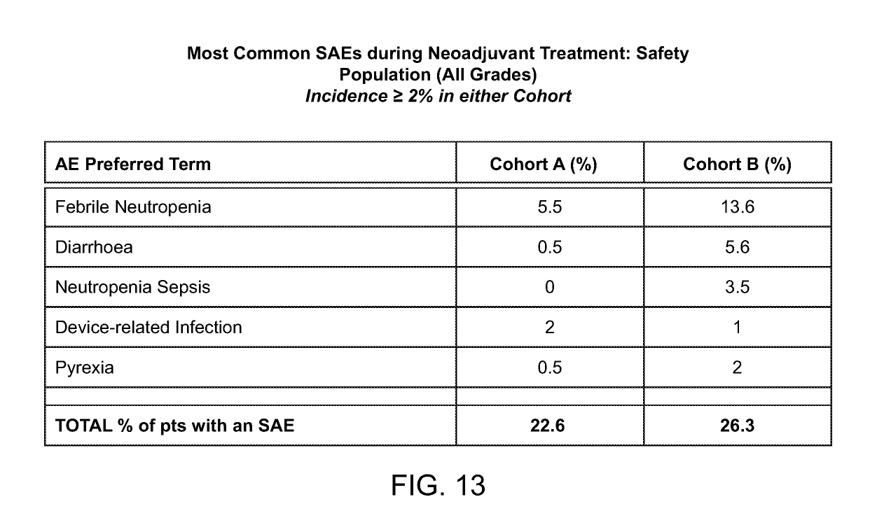

FIG. 13 lists the most common serious adverse events (SAEs) during neoadjuvant treatment in the safety population (All Grades). Incidence ≥2% in either Cohort. Febrile neutropenia, diarrhea, neutropenia sepsis, device-related infection, and pyrexia are included.

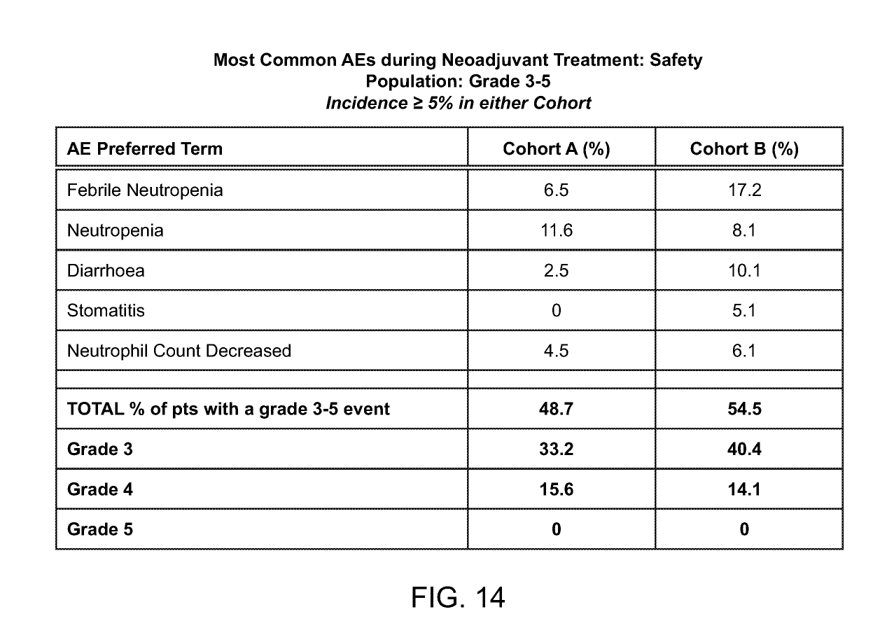

FIG. 14 lists the most common AEs during neoadjuvant treatment: Safety Population: Grade 3-5. Incidence ≥5% in either Cohort. Febrile neutropenia, neutropenia, diarrhea, stomatitis, neutrophil count decreased are included.

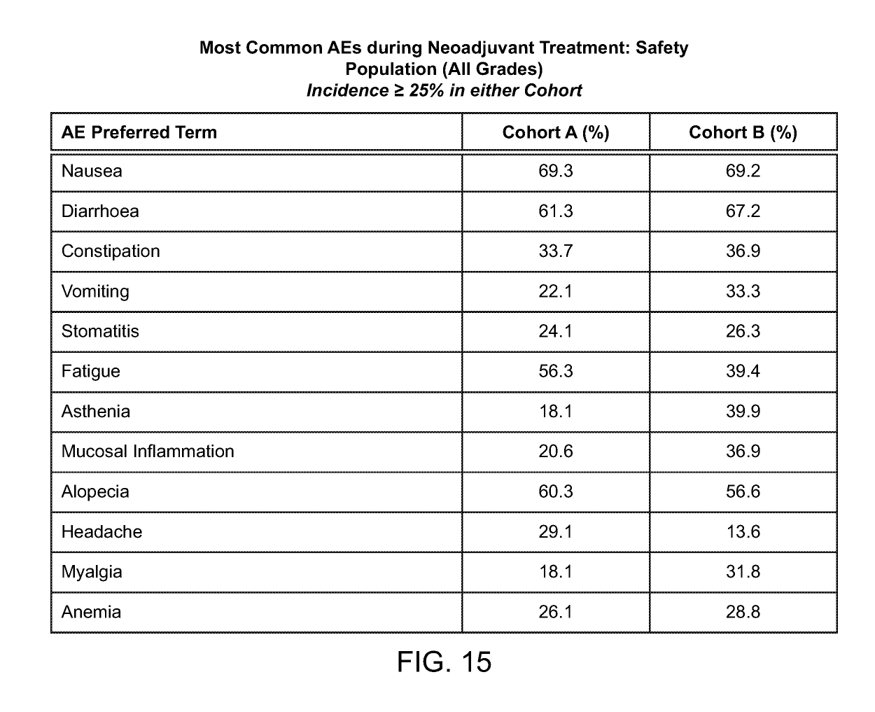

FIG. 15 lists the most common AEs during neoadjuvant treatment: Safety Population (All Grades). Incidence ≥25% in either Cohort. Nausea, diarrhea, constipation, vomiting, stomatitis, fatigue, asthenia, mucosal inflammation, alopecia, headache, myalgia, and anemia are included.

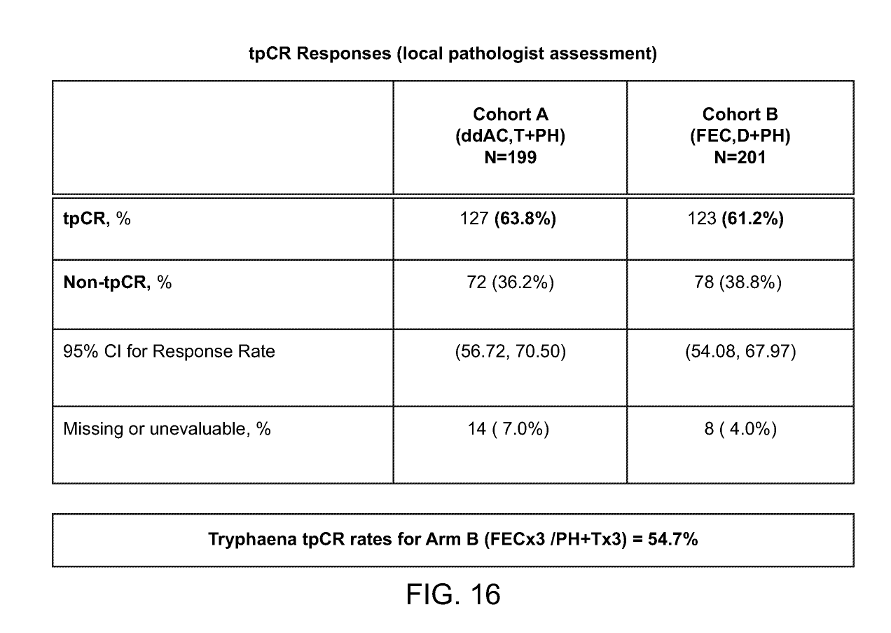

FIG. 16 is a summary of tpCR responses based on assessment by the local pathologist. In Cohort A the response rate (tpCR) was 63.8%, and in Cohort B the response rate (tpCR) was 61.2%.

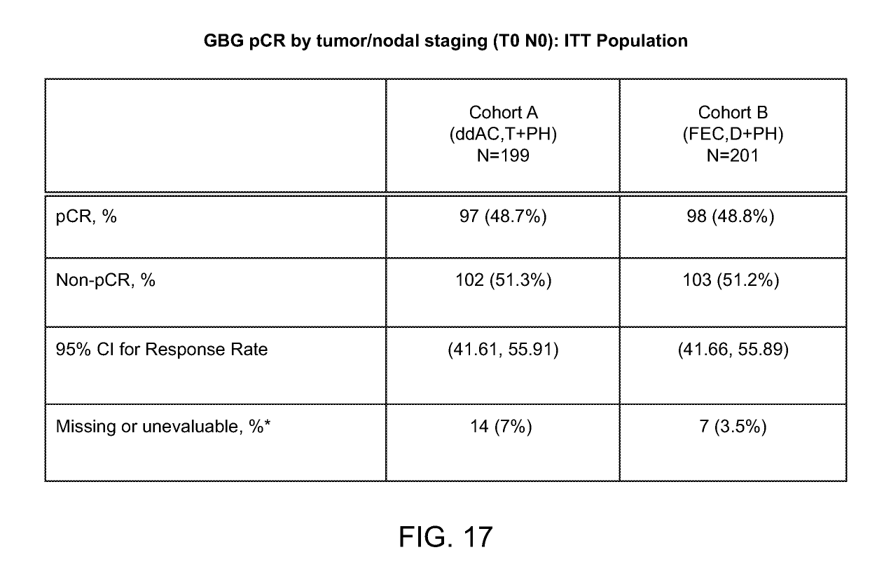

FIG. 17 shows pCR response rate in the German Breast Group (GBG) by tumor/nodal staging (TO NO): Intention-To-Treat (ITT) population. In Cohort A the response rate (pCR) was 48.7% and in Cohort B the response rate (pCR) was 48.8%.

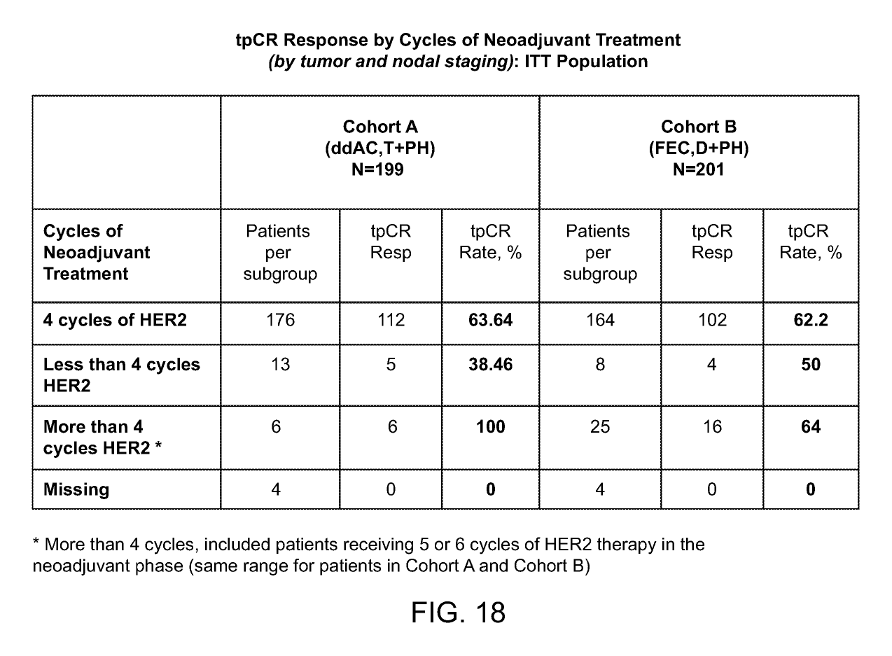

FIG. 18 shows tpCR response by cycles of neoadjuvant treatment (by tumor and nodal staging): Intention-To-Treat (ITT) population, including 4 cycles, less than 4 cycles and more than 4 cycles of neoadjuvant treatment.

Biomarkers show that majority of patients are able to be assessed for PAM50 sub-type and pCR rates are consistent with HR subgroup analysis. The majority of patients were categorized as HER2 enriched subtype (39.7% [n 79] in Cohort A and 47.3% [in 95] in Cohort B). Luminal A subtypes were identified in 16.6% (n 33) in Cohort A and 15.4% (n 31) in Cohort B, and Luminal B subtypes were identified in 12.1% n 24) of patients in Cohort A and 7.5% (n 15) in Cohort B.

Slight imbalances in distribution of the individual subtypes between the two cohorts were observed for HER2 enriched and Luminal B subtypes. The highest tpCR rate was observed in the HER2 enriched subgroup with 76.0% (n 60) and 73.7% (n 70). The tpCR rates in the subgroups of Luminal A and Luminal B were comparable (range: 42%-46%).

The lower tpCR rate observed in the luminal subgroups resembles the lower tpCR rate observed in the ER positive subgroups defined by central ER status per IHC.

### REFERENCES

Andrulis I L, Bull S B, Blackstein M E, et al., for the Toronto Breast Cancer Study Group. Neu/erbB-2 amplification identifies a poor prognosis group of women with node-negative breast cancer. J Clin Oncol 1998; 16:1340-9.
Baselga J, Bradbury I, Eidtmann H, et al. Lapatinib with Trastuzumab for HER2-positive early breast cancer (NeoALTTO): a randomised, open-label, multicentre, phase 3 trial. Lancet 2012a; 379(9816):633-40.
Baselga J, Cortes J, Kim S B, et al., for the CLEOPATRA Study Group. Perjeta plus Herceptin plus docetaxel for metastatic breast cancer. N Engl J Med 2012b; 366:109-19.
Bear H D, Anderson S, Smith R E, et al. Sequential preoperative or postoperative docetaxel added to preoperative doxorubicin plus cyclophosphamide for operable breast cancer: National Surgical Adjuvant Breast and Bowel Project Protocol B-27. J Clin Oncol 2006; 24:2019-27.
Berruti A, Generali D, Kaufmann M, et al. International expert consensus on primary systemic therapy in the management of early breast cancer: highlights of the Fourth Symposium on Primary Systemic Therapy in the Management of Operable Breast Cancer, Cremona, Italy. J Natl Cancer Inst Monogr 2011; 43:147-51.
Buzdar A U, Ibrahim N K, Francis D, et al. Significantly higher pathologic complete remission rate after neoadjuvant therapy with Trastuzumab, paclitaxel, and epirubicin chemotherapy: results of a randomized trial in human epidermal growth factor receptor 2-positive operable breast cancer. J Clin Oncol 2005; 23:3676-85.
Buzdar A U, Valero V, Ibrahim N K, et al. Neoadjuvant therapy with paclitaxel followed by 5-fluorouracil, epirubicin, and cyclophosphamide chemotherapy and concurrent Trastuzumab in human epidermal growth factor receptor 2-positive operable breast cancer: an update of the initial randomized study population and data of additional patients treated with the same regimen. Clin Cancer Res 2007; 13:228-33.
Carey L A, Berry D A, Ollila D, et al. Clinical and translational results of CALGB 40601: A neoadjuvant phase III trial of weekly paclitaxel and Trastuzumab with or without lapatinib for HER2-positive breast cancer. J Clin Oncol 2013; 31(suppl abstr):500).
Cho H S, Mason K, Ramyar K X, et al. Structure of the extracellular region of HER2 alone and in complex with the Herceptin Fab. Nature 2003; 421(6924):756-60.
Citron M L, Berry D A, Cirrincione C, et al. Randomized trial of dose-dense versus conventionally scheduled and sequential versus concurrent combination chemotherapy as postoperative adjuvant treatment of node-positive primary breast cancer: first report of Intergroup Trial C9741/Cancer and Leukemia Group B Trial 9741. J Clin Oncol 2003; 21:1431-9.
Cortazar P, Zhang L, Untch M, et al. Metaanalysis results from the Collaborative Trials in Neoadjuvant Breast Cancer (CTNeoBC) [abstract]. Cancer Res 2012; 72(24 Suppl.)93s:S1-11.
Dang C, Fornier M, Sugarman S, et al. The safety of dose-dense doxorubicin and cyclophosphamide followed by paclitaxel with Trastuzumab in HER-2/neu overexpressed/amplified breast cancer. J Clin Oncol 2008; 26:1216-22.
Datko F M, D'Andrea G, Dickler M N et al. Phase II study of Pertuzumab, Trastuzumab, and weekly paclitaxel in patients with HER2-overexpressing metastatic breast cancer (MBC). J Clin Oncol 31 2013; (suppl; abstr 606).
Dawood S, Merajver S D, Viens P, et al. International expert panel on inflammatory breast cancer: consensus statement for standardized diagnosis and treatment. Ann Oncol 2011; 22:515-23.
Dent S, Oyan B, Honig A, et al. HER2-targeted therapy in breast cancer: a systematic review of neoadjuvant trials. Cancer Treat Rev. 2013; 39:622-31.
Early Breast Cancer Trialists' Collaborative Group (EBCTCG). Effect of radiotherapy after breast-conserving surgery on 10-year recurrence and 15-year breast cancer death: meta-analysis of individual patient data for 10,801 women in 17 randomised trials. Lancet 2011a; 378:1707-16.
Early Breast Cancer Trialists' Collaborative Group (EBCTCG). Relevance of breast cancer hormone receptors and other factors to the efficacy of adjuvant tamoxifen: patient-level meta-analysis of randomised trials. Lancet 2011b; 378:771-84.
Eisenhauer A E, Therasse P, Bogaerts J, et al. New response evaluation criteria in solid tumours: Revised RECIST guideline (version 1.1). Eur J Cancer 2009; 45:228-47.
Esserman L J, Berry D A, DeMichele A, et al. Pathologic complete response predicts recurrence-free survival more effectively by cancer subset: results from the I-SPY 1 TRIAL-CALGB 150007/150012, ACRIN 6657. J Clin Oncol 2012; 30:3242-9.
Ferlay J, Shin H R, Bray F, et al. Estimates of worldwide burden of cancer in 2008: GLOBOCAN 2008. Int J Cancer 2010; 127:2893-917.
Franklin M C, Carey K D, Vajdos F F, et al. Insights into ErbB signaling from the structure of the ErbB2-Pertuzumab complex. Cancer Cell 2004; 5:317-28.
Gianni L, Eiermann W, Semiglazov V, et al. Neoadjuvant chemotherapy with Trastuzumab followed by adjuvant Trastuzumab versus neoadjuvant chemotherapy alone, in patients with HER2-positive locally advanced breast cancer: a randomised controlled superiority trial with a parallel HER2-negative cohort (the NOAH trial). Lancet 2010; 375:377-84.
Gianni L, Pienkowski T, Im Y H, et al. Efficacy and safety of neoadjuvant Pertuzumab and Trastuzumab in women with locally advanced, inflammatory, or early HER2-positive breast cancer (NeoSphere): a randomised multicentre, open-label, phase 2 trial. Lancet Oncol 2012; 13:25-32.
Gianni L, Eiermann W, Semiglazov V, et al. Follow-up results of NOAH, a randomized phase III trial evaluating neoadjuvant chemotherapy with Trastuzumab (CT+H) followed by adjuvant H versus C T alone, in patients with HER2-positive locally advanced breast cancer. J Clin Oncol 2013; 31(15 suppl; abstr 503).
Guarneri V, Frassoldati A, Bottini A, et al. Preoperative chemotherapy plus Trastuzumab, lapatinib, or both in human epidermal growth factor receptor 2-positive operable breast cancer: results of the randomized phase II CHER-LOB study. J Clin Oncol 2012; 30:1989-95.
Hammond M E H, Hayes D F, Dowsett M, et al. American Society of Clinical Oncology/College of American Pathologists guideline recommendations for immunohistochemical testing of estrogen and progesterone receptors in breast cancer. J Clin Oncol 2010; 28:2784-95.
Hanahan D, Weinberg R A. The hallmarks of cancer. Cell 2000; 100:57-70.
Hance K W, Anderson W F, Devesa S S, et al. Trends in inflammatory breast carcinoma incidence and survival: the surveillance, epidemiology, and end results program at the National Cancer Institute. J Natl Cancer Inst 2005; 97:966-75.
Harbeck N, Thomssen C, Gnant M. St. Gallen 2013: Brief preliminary summary of the consensus discussion. Breast Care 2013; 8:102-9.
Howlader N, Noone A M, Krapcho M, et al. SEER cancer statistics review, 1975-2008 [Internet]. Bethesda, Md. National Cancer Institute; November 2010 [updated, 2011]. Available from: http://seer.cancer.gov/csr/1975_2008/
Hudis C, Citron M L, Berry D. Five year follow-up of INT C9741: dose dense chemotherapy is safe and effective. Breast Cancer Res 2005; 94(Suppl 1):520.
Hudis C A, Barlow W E, Costantino J P, et al. Proposal for standardized definitions for efficacy end points in adjuvant breast cancer trials: the STEEP system. J Clin Oncol 2007; 25:2127-32.
Ismael G, Hegg R, Muehlbauer S, et al. Subcutaneous versus intravenous administration of (neo)adjuvant Trastuzumab in patients with HER2-positive, clinical stage I-III breast cancer (HannaH study): phase 3, open-label, muticentre, randomized trial. Lancet Oncol 2012; 13:869-78.
Jemal A, Bray F, Center M M, et al. Global cancer statistics. C A Cancer J Clin 2011; 61:69-90.
Kim M M, Allen P, Gonzalez-Angulo A M, et al. Pathologic complete response to neoadjuvant chemotherapy with Trastuzumab predicts for improved survival in women with HER2-overexpressing breast cancer. Ann Oncol 2013; 24:1999-2004.
Kong X, Moran M S, Zhang N, et al. Meta-analysis confirms achieving pathological complete response after neoadjuvant chemotherapy predicts favourable prognosis for breast cancer patients. Eur J Cancer 2011; 47:2084-90.
Kwan M L, Kushi L H, Weltzien E, et al. Epidemiology of breast cancer subtypes in two prospective cohort studies of breast cancer survivors. Breast Cancer Res 2009; 11:R31.
Levi F, Bosetti C, Lucchini F, et al. Monitoring the decrease in breast cancer mortality in Europe. Eur J Cancer Prev 2005; 14:497-502.
Malvezzi M, Bertuccio P, Levi F, et al. European cancer mortality predictions for the year 2013. Ann Oncol 2013; 24:792-800.
Mamounas E P, Anderson S J, Dignam J J, et al. Predictors of locoregional recurrence after neoadjuvant chemotherapy: results from combined analysis of National Surgical Adjuvant Breast and Bowel Project B-18 and B-27. J Clin Oncol 2012; 30:3960-6.
Mauri D, Pavlidis N, Ioannidis J P. Neoadjuvant versus adjuvant systemic treatment in breast cancer: a meta-analysis. J Natl Cancer Inst 2005; 97:188-94.
Mazouni C, Peintinger F, Wan-Kau S, et al. Residual ductal carcinoma in situ in patients with complete eradication of invasive breast cancer after neoadjuvant chemotherapy does not adversely affect patient outcome. J Clin Oncol 2007; 25:2650-5.
Mieog J S, van der Hage J A, van de Velde C J. Preoperative chemotherapy for women with operable breast cancer. Cochrane Database Syst Rev 2007; 18:CD005002.
Moja L, Tagliabue L, Balduzzi S, et al. Trastuzumab containing regimens for early breast cancer. Cochrane Database Syst Rev 2012; 4:CD006243.
Morris P G, lyengar N M, Patil S, et al. Long-term cardiac safety and outcomes of dose-dense doxorubicin and cyclophosphamide followed by paclitaxel and Trastuzumab with and without lapatinib in patients with early breast cancer. Cancer 2013; 119:3943-51.
Neven P, Van Calster A E I, Van den Bempt A E, et al. Age interacts with the expression of steroid and HER-2 receptors in operable invasive breast cancer. Breast Cancer Res Treat 2008; 110:153-9.
Oken M M, Creech R H, Tormey D C, et al. Toxicity and response criteria of the Eastern Cooperative Oncology Group. Am J Clin Oncol 1982; 5:649-55.
Olayioye M A, Neve R M, Lane H A, et al. The erbB signaling network: receptor heterodimerization in development and cancer. EMBO J 2000; 19:3159-67.
Parker J S, Mullins M, Cheang M C, et al. Supervised risk predictor of breast cancer based on intrinsic subtypes. J Clin Oncol 2009; 27:1160-7.
Pauletti G, Dandekar S, Rong H, et al. Assessment of methods for tissue-based detection of the HER-2/neu alteration in human breast cancer: a direct comparison of fluorescence in situ hybridization and immunohistochemistry. J Clin Oncol 2000; 18:3651-64.
Perez E A, Suman V J, Davidson N E, et al. Cardiac safety analysis of doxorubicin and cyclophosphamide followed by paclitaxel with or without Trastuzumab in the North Central Cancer Treatment Group N9831 adjuvant breast cancer trial. J Clin Oncol 2008; 26:1231-8.
Rastogi P, Anderson S J, Bear H D, et al. Preoperative chemotherapy: updates of National Surgical Adjuvant Breast and Bowel Project Protocols B-18 and B-27. J Clin Oncol 2008; 26:778-85.
Robidoux A, Tang G, Rastogi P, et al. Lapatinib as a component of neoadjuvant therapy for HER2-positive operable breast cancer (NSABP protocol B-41): an open-label, randomised phase 3 trial. Lancet Oncol 2013; 14:1183-92.
Romond E, Perez E A, Bryant J, et al. Trastuzumab plus adjuvant chemotherapy for operable HER2-positive breast cancer. N Engl J Med 2005; 353:1673-84.
Romond E H, Jeong J-H, Rastogi P, et al. Seven-year follow-up assessment of cardiac function in NSABP B-31, a randomized trial comparing doxorubicin and cyclophosphamide followed by paclitaxel (ACP) with ACP plus Trastuzumab as adjuvant rherapy for patients with node-positive, human epidermal growth factor receptor 2-positive breast cancer. J Clin Oncol 2012; 30:3792-9.
Rubin I, Yarden Y. The basic biology of HER2. Ann Oncol 2001; 12(Suppl 1):53-8.
Russell S D, Blackwell K L, Lawrence J, et al. Independent adjudication of symptomatic heart failure with the use of doxorubicin and cyclophosphamide followed by Trastuzumab adjuvant therapy: a combined review of cardiac data from the National Surgical Adjuvant Breast and Bowel Project B-31 and the North Central Cancer Treatment Group N9831 clinical trials. J Clin Oncol 2010; 28:3416-21.
Sant M, Allemani C, Capocaccia R, et al. Stage at diagnosis is a key explanation of differences in breast cancer survival across Europe. Int J Cancer 2003; 106:416-22.
Sawyer D B, Zuppinger C, Miller T A, et al. Modulation of anthracycline-induced myofibrillar disarray in rat ventricular myocytes by neuregulin-1 beta and anti-erbB2: Potential mechanism for Trastuzumab-associated cardiotoxicity. Circulation 2002; 105:1551-4.
Schneeweiss A, Chia S, Hickish T, et al. Pertuzumab plus Trastuzumab in combination with standard neoadjuvant anthracycline-containing and anthracycline-free chemotherapy regimens in patients with HER2-positive early breast cancer: a randomized phase II cardiac safety study (TRYPHAENA). Ann Oncol 2013; 24:2278-84.
Senkus E, Kyriakides S, Penault-Llorca F, et al. Primary breast cancer: ESMO Clinical Practice Guidelines for diagnosis, treatment and follow-up. Ann Oncol 2013; 24(Suppl 6):vi7-23.

What is claimed is:

1. A method for the treatment of breast cancer comprising neoadjuvant administration to a patient with HER2-positive locally advanced, inflammatory, or early-stage breast cancer of an effective amount of a combination of pertuzumab and trastuzumab following anthracycline-based chemotherapy, wherein the combined administration of pertuzumab and trastuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.

2. The method of any one of claim 1, wherein the combined administration of pertuzumab and trastuzumab starts after at least 4 cycles of anthracycline-based chemotherapy.

3. The method of claim 1, wherein the anthracycline-based chemotherapy comprises doxorubicin.

4. The method of claim 3, wherein the anthracycline-based chemotherapy comprises doxorubicin plus cyclophosphamide.

5. The method of claim 4, wherein the anthracycline-based chemotherapy is doxorubicin plus cyclophosphamide (AC).

6. The method of claim 4, wherein the anthracycline-based chemotherapy is dose-dense doxorubicin and cyclophosphamide (ddAC).

7. The method of any one of claims 4 to 6, wherein doxorubicin plus cyclophosphamide are administered with G-CSF support.

8. The method of any one of claims 4 to 6, wherein the anthracycline-based chemotherapy is administered every two weeks.

9. The method of any one of claims 4 to 8, wherein at least four cycles of the anthracycline-based chemotherapy are administered prior to the combined administration of pertuzumab and trastuzumab.

10. The method of claim 1, wherein the anthracycline-based chemotherapy comprises epirubicin.

11. The method of claim 10, wherein the anthracycline-based chemotherapy comprises epirubicin, 5-fluorouracil and cyclophosphamide.

12. The method of claim 11, wherein the anthracycline-based chemotherapy is 5-fluorouracil, epirubicin plus cyclophosphamide (FEC).

13. The method of any one of claims 10 to 12, wherein the anthracycline-based chemotherapy is administered every three weeks.

14. The method of any one of claims 10 to 13, wherein at least four cycles of the anthracycline-based chemotherapy are administered prior to the combined administration of pertuzumab and trastuzumab.

15. The method of any one of claims 1 to 14, wherein pertuzumab and trastuzumab are administered in combination with neoadjuvant administration of a taxane.

16. The method of claim 15, wherein the taxane is docetaxel.

17. The method of claim 15, wherein the taxane is paclitaxel.

18. The method of any one of claims 15 to 17, wherein the combined administration of pertuzumab and trastuzumab starts at the start of taxane administration.

19. The method of any one of claims 1 to 18, wherein the pCR is breast pathologic complete response (bpCR).

20. The method of any one of claims 1 to 18, wherein the pCR is total pathologic complete response (tpCR).

21. The method of any one of claims 1 to 20, wherein the adverse events include cardiac side-effects.

22. The method of any one of claims 1 to 20, wherein the adverse event is a cardiac side-effect.

23. The method of claim 21 or 22, wherein the cardiac side-effect comprises left ventricular ejection fraction (LVEF) drop.

24. The method of claim 23, wherein the LVEF drop is asymptomatic.

25. The method of claim 21 or 22, wherein the cardiac side-effect comprises left ventricular systolic dysfunction (LVSD).

26. The method of claim 25, wherein the LVSD is symptomatic.

27. The method of any one of claims 1 to 26, wherein the HER2-positive breast cancer is characterized by immunohistochemistry (IHC) score 3+ or 2+ or by an amplification ratio of ≥2.0 determined by fluorescence in situ hybridization.

28. The method of any one of claims 1 to 27, wherein the HER2-positive breast cancer is of Luminal A, Luminal B, HER2-Enriched (HER2-E) or Basal-like subtype as determined by PAM50 RT-qPCR assay.

29. The method of claim 28, wherein the HER2-positive breast cancer is HER2-E subtype.

30. The method of any one of claims 1 to 27, wherein the HER2-positive breast cancer is characterized by aberrant PI3K pathway.

31. The method of any one of claims 1 to 27, wherein the HER2-positive breast cancer is acetyltanshinone IIA (ATA) positive.

32. The method of any one of claims 1 to 31, wherein the neoadjuvant administration is followed by definitive surgery.

33. The method of claim 32, wherein definitive surgery is performed after at least eight cycles of neoadjuvant therapy.

34. The method of claim 32 or 33, wherein definitive surgery is followed by adjuvant administration of pertuzumab plus trastuzumab.

35. The method of any one of claims 1 to 34, wherein pCR correlates with progression-free survival (PFS).

36. A method for extending the pathological complete response (pCR) in a patient with HER2-positive, locally advanced, inflammatory, or early-stage breast cancer by neoadjuvant administration of a combination of pertuzumab and trastuzumab following anthracycline-based chemotherapy, relative to administration of trastuzumab following anthracycline-containing chemotherapy, without significant increase in adverse events relative to neoadjuvant anthracycline-containing chemotherapy.

37. An article of manufacture comprising a vial with pertuzumab and a package insert, wherein the package insert provides at least part of the safety data shown in FIGS. 10-15.

38. The article of manufacture of claim 37 which comprises a single-dose vial containing about 420 mg of pertuzumab.

39. A method for making an article of manufacture comprising packaging together a vial with pertuzumab therein and a package insert, wherein the package insert provides at least part of the safety data shown in FIGS. 10-15.

40. A method of ensuring safe and effective use of pertuzumab comprising packaging together a vial with pertuzumab therein and a package insert, wherein the package insert provides at least part of the safety data shown in FIGS. 10-15.

41. Use of pertuzumab in the preparation of a medicament for treatment of breast cancer in a patient with HER2-positive locally advanced, inflammatory, or early-stage breast cancer comprising neoadjuvant administration of an effective amount of a combination of said pertuzumab and trastuzumab following anthracycline-based chemotherapy, wherein the combined administration of pertuzumab and trastuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.

42. Pertuzumab for use in the treatment of breast cancer in a patient with HER2-positive locally advanced, inflammatory, or early-stage breast cancer, wherein said treatment comprises neoadjuvant administration of an effective amount of a combination of said pertuzumab and trastuzumab following anthracycline-based chemotherapy, wherein the combined administration of pertuzumab and trastuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.

43. Use of trastuzumab in the preparation of a medicament for treatment of breast cancer in a patient with HER2-positive locally advanced, inflammatory, or early-stage breast cancer comprising neoadjuvant administration of an effective amount of a combination of said trastuzumab and pertuzumab following anthracycline-based chemotherapy, wherein the combined administration of pertuzumab and trastuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.

44. Trastuzumab for use in the treatment of breast cancer in a patient with HER2-positive locally advanced, inflammatory, or early-stage breast cancer, wherein said treatment comprises neoadjuvant administration of an effective amount of a combination of said trastuzumab and pertuzumab following anthracycline-based chemotherapy, wherein the combined administration of trastuzumab and pertuzumab following anthracycline-based chemotherapy increases pathological complete response (pCR) relative to administration of trastuzumab following anthracycline-based chemotherapy, without significant increase in adverse events relative to neoadjuvant anthracycline-based chemotherapy.


SEQUENCE LISTING (22 sequences)

SEQ ID NO: 1 - protein, 195 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTR

SEQ ID NO: 2 - protein, 124 aa, Homo sapiens
TVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARV

SEQ ID NO: 3 - protein, 169 aa, Homo sapiens
CYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLA

SEQ ID NO: 4 - protein, 142 aa, Homo sapiens
CHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 5 - protein, 107 aa, Mus musculus
DTVMTQSHKIMSTSVGDRVSITCKASQDVSIGVAWYQQRPGQSPKLLIYSASYRYTGVPDRFTGSGSGTDFTFTISSVQAEDLAVYYCQQYYIYPYTFGGGTKLEIK

SEQ ID NO: 6 - protein, 119 aa, Mus musculus
EVQLQQSGPELVKPGTSVKISCKASGFTFTDYTMDWVKQSHGKSLEWIGDVNPNSGGSIYNQRFKGKASLTVDRSSRIVYMELRSLTFEDTAVYYCARNLGPSFYFDYWGQGTTLTVSS

SEQ ID NO: 7 - protein, 107 aa, Artificial Sequence [sequence omitted]

SEQ ID NO: 8 - protein, 119 aa, Artificial Sequence [sequence omitted]

SEQ ID NO: 9 - protein, 107 aa, Artificial Sequence
  Description of Artificial Sequence Synthetic polypeptide
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIK

SEQ ID NO: 10 - protein, 119 aa, Artificial Sequence
  Description of Artificial Sequence Synthetic polypeptide
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 11 - protein, 214 aa, Artificial Sequence
  Description of Artificial Sequence Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 448 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 217 aa, Artificial Sequence
  Description of Artificial Sequence Synthetic polypeptide
VHSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 449 aa, Artificial Sequence
  Description of Artificial Sequence Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 10 aa, Artificial Sequence
  Description of Artificial Sequence Synthetic peptide
  MOD_RES (10)..(10): Asp or Ser
GFTFTDYTMX

SEQ ID NO: 18 - protein, 17 aa, Artificial Sequence
  Description of Artificial Sequence Synthetic peptide
DVNPNSGGSIYNQRFKG

SEQ ID NO: 19 - protein, 10 aa, Artificial Sequence
  Description of Artificial Sequence Synthetic peptide
NLGPSFYFDY

SEQ ID NO: 20 - protein, 11 aa, Artificial Sequence
  Description of Artificial Sequence Synthetic peptide
KASQDVSIGVA

SEQ ID NO: 21 - protein, 7 aa, Artificial Sequence
  Description of Artificial Sequence Synthetic peptide
  MOD_RES (5)..(5): Arg or Leu
  MOD_RES (6)..(6): Tyr or Glu
  MOD_RES (7)..(7): Thr or Ser
SASYXXX

SEQ ID NO: 22 - protein, 9 aa, Artificial Sequence
  Description of Artificial Sequence Synthetic peptide
QQYYIYPYT